(19) United States
(12) Patent Application Publication
(10) Pub. No.: US 2022/0257219 A1
(43) Pub. Date: Aug. 18, 2022

(54) METHOD OF COLLECTING OROPHARYNGEAL LAVAGE, IN VITRO IMMUNOCHROMATOGRAPHIC ASSAY, AND COMPOSITION AND KIT THEREFOR

(71) Applicant: Zeus Diagnostics, LLC, Severna Park, MD (US)

(72) Inventor: Raouf A. Guirguis, Fond Du Lac, WI (US)

(21) Appl. No.: 17/670,960

(22) Filed: Feb. 14, 2022

Related U.S. Application Data
(60) Provisional application No. 63/200,079, filed on Feb. 12, 2021.

(51) Int. Cl. A61B 10/00; B01L 3/02; B01L 3/00

(52) U.S. Cl. CPC A61B 10/0051; B01L 3/5029; B01L 3/0272

(57) ABSTRACT

A method for collecting a liquid sample from the oral cavity and oropharyngeal area comprises in part: pouring a non-alcoholic mouth rinse into the oral cavity; using a collection swab comprising a sponge head to scrub the inside of the oral cavity; and inserting the collection swab under the tongue to absorb the mouth rinse. The non-alcoholic mouth rinse is a composition comprising: a therapeutic antimicrobial mouth rinse; a salt; and a mucolytic agent. An in vitro immunochromatographic assay comprises in part: collecting a liquid sample from the oral cavity and oropharyngeal area; adding a portion of the liquid sample to the sample port of a test card capable of immunochromatographic assay; and viewing the results on the test card. The assay is applicable to the qualitative detection of SARS-CoV-2 virus antigen. A kit for the qualitative detection of SARS-CoV-2 virus antigen is also disclosed.

### CROSS REFERENCE TO RELATED APPLICATION

This non-provisional patent application claims benefit of U.S. Provisional Application No. 63/200,079 filed Feb. 12, 2021 and is incorporated herein by reference in its entirety.

### BACKGROUND

This disclosure relates to methods of collecting oropharyngeal lavage, a composition for collecting oropharyngeal lavage, and in vitro immunochromatographic assays and test kits for detecting one or more analytes in oropharyngeal lavage. The disclosure also relates to a rapid in vitro immunochromatographic assay and test kits for the qualitative detection of SARS-CoV-2 virus antigen present in human oropharyngeal lavage.

SARS-CoV-2 is single-stranded RNA virus with an envelope (virion). It is approximately 50 to 200 nanometers in diameter. It has four structural proteins, known as the spike (S), envelope (E), membrane (M), and nucleocapsid (N) proteins. The N protein holds the RNA genome, and the S, E, and M proteins together create the viral envelope. The incubation period for COVID-19 typically ranges from 2 to 14 days. Those infected with the virus may be asymptomatic or develop common respiratory symptoms, including fever, cough and fatigue. Other symptoms may include muscle pain, diarrhea, sore throat, loss of taste and smell, and abdominal pain. Severe cases may progress to acute respiratory distress syndrome (ARDS), septic shock, diffuse alveolar damage (DAD), and even death. Conventional in vitro immunochromatographic assays for the qualitative detection of SARS-CoV-2 virus antigen require nasopharyngeal swabs (NP). Although these tests have proven a useful tool for the detection of SARS-CoV-2 and other respiratory infections, the use of a nasopharyngeal (NP) swab for sample collection is highly problematic. First, nasopharyngeal swabs should only be performed by trained medical personnel. Therefore, tests requiring NP swabs are not recommended for home use. Moreover, sample collection by NP swab can be extremely uncomfortable. Home tests utilize a nasal swab (of nostrils), which can be performed by the subject at home, and are less uncomfortable. However, the nasal swab can be prone to false negatives, especially in patients with low SARS-CoV-2 viral loads. The nasal swab can also be prone to false positives, because viral particles can be trapped in the nostrils without causing infection.

There remains a need in the medical arts for an in vitro immunochromatographic assay for the qualitative detection of SARS-CoV-2 virus antigen that can be performed at home by the test subject or family member, that is comfortable, and that is not subject to false negatives or false positives. It can also be desirable that an assay be able to detect the virus antigen early, especially before the onset of symptoms, which can take five days or more to appear.

### SUMMARY

A method for collecting a liquid sample from the oral cavity and oropharyngeal area comprises: avoiding placing food, drink, gum, or tobacco products in the oral cavity for a least twenty minutes prior to collecting the liquid sample; pouring a non-alcoholic mouth rinse into the oral cavity; swishing and gargling the mouth rinse at least once to sweep inside of the oral cavity, including the inside of the cheeks, the tongue, and back of the throat, while not spitting or swallowing the mouth rinse; using a collection swab comprising a sponge head to scrub the inside of the oral cavity, including the inside of the cheeks, the tongue and back of the throat; inserting the collection swab under the tongue; holding the collection swab under the tongue for at least one minute without biting, sucking, or chewing on the sponge head to absorb the mouth rinse; and removing the collection swab from the oral cavity; wherein the sponge head is designed to absorb and discharge the entire volume of the mouth rinse. A related method for collecting a liquid sample from the oral cavity and oropharyngeal area of a human child is also disclosed. The non-alcoholic mouth rinse is a composition comprising: a therapeutic antimicrobial mouth rinse; a salt; and a mucolytic agent.

In other embodiments, a rapid in vitro immunochromatographic assay for the qualitative detection of analytes present in human oropharyngeal lavage comprises: collecting a liquid sample from the oral cavity and oropharyngeal area; adding a portion of the liquid sample to the sample port of a test card capable of immunochromatographic assay; and viewing the results on the test card; wherein a first colored band on the test card indicates a positive test result and a second colored control band indicates a valid test result. In some embodiments, the rapid in vitro immunochromatographic assay is for the qualitative detection of SARS-CoV-2 virus antigen present in human oropharyngeal lavage. This assay comprises: collecting a liquid sample from the oral cavity and oropharyngeal area; adding a portion of the liquid sample to the sample port of a test card capable of detecting the SARS-CoV-2 virus antigen; and viewing test results on the test card; wherein a first colored band on the test card indicates a positive test result and a second colored control band indicates a valid test result.

A kit for rapid in vitro immunochromatographic assay for the qualitative detection of SARS-CoV-2 virus antigen present in human oropharyngeal lavage is also disclosed. The kit comprises: a collection swab comprising a sponge head; a non-alcoholic mouth rinse; a sample buffer; a sample collector; a cap for the sample collector; a test card capable of detecting the SARS-CoV-2 virus antigen; and instructions for conducting the assay.

### BRIEF DESCRIPTION OF THE DRAWINGS

FIG. 1 is a drawing of a kit 100, including test card 10, sample collector, sachet of non-alcoholic mouth rinse 14, 16, cap 18, collection swab 20 including sponge head 22, and packaging 17.

FIG. 2a to 2d are schematic views illustrating the method for collecting a liquid sample.

FIG. 2a illustrates schematically no smoking, no eating and drinking, and no chewing gum.

FIG. 2b illustrates schematically pouring a non-alcoholic mouth rinse 14 into the mouth, swishing and gargling the mouth rinse at least once to sweep inside of the mouth, including the inside of the cheeks, the tongue, and back of the throat, while not spitting or swallowing the mouth rinse solution.

FIG. 2c illustrates schematically using a collection swab 20 comprising a sponge head 22 to scrub the inside of the mouth, including the inside of the cheeks, the tongue and back of the throat, and to absorb the mouth rinse; inserting the collection swab 20 under the tongue; and holding the collection swab 20 under the tongue for at least one minute without biting, sucking, or chewing on the collection sponge.

FIG. 2d illustrates inserting the collection swab 20, sponge-end first, into a sample collector 16 wherein at least a section of the sample collector 16 has an inner diameter or width smaller than the width of the sponge head 22; pushing the sponge head 22 of the collection swab 20 up and down into the space defined by the inner wall of the section of the sample collector 16 at least three times; and sealing the sample collector 16 with a cap 18.

FIG. 3a to 3g are schematic views illustrating the in vitro immunochromatographic assay.

FIG. 3a illustrates checking test card 10 expiration date.

FIG. 3b illustrates bringing the kit components to room temperature.

FIG. 3c illustrates removing test card 10 from its packaging.

FIG. 3d illustrates labelling the test device with a test identification number (ID).

FIG. 3e illustrates moving nozzle in closed position 38 to open position 39.

FIG. 3f illustrates adding a portion of the liquid sample to sample port 30 of test card 10.

FIG. 3g illustrates first colored band 40 of test card 10 indicating a positive test result and second colored band 41 indicating the test result is valid.

FIG. 4a to 4d illustrate various test card 10 results.

FIG. 4a illustrates a first colored band 40 indicating a positive test result and a second colored band 41 indicating a valid test result.

FIG. 4b illustrates the absence of a first colored band 40 indicating a negative test result and second colored band 41 indicating a valid test result.

FIG. 4c illustrates a first colored band 40 indicating a positive test result and the absence a second colored band 41 indicating an invalid test result.

FIG. 4d illustrates the absence of a first colored band 40 indicating a negative test result and the absence a second colored band 41 indicating an invalid test result.

FIG. 5 is a photo of sample collector 16, cap 18, extraction buffer 12, and collection swab 20 with sponge head 22, test kit components.

FIG. 6 is a schematic plot of SARS-CoV-2 RNA and antigen, IgM antibody, and IgG antibody amounts as a function of days since infection. FIG. 6 also indicates the asymptomatic stage (0 to 5 days), the onset of symptoms (ca. 5 to 8 days), decline (recovery) stage (ca. 13 to 20 days), and convalescent stage (ca. 20 to 28 days), all delineated by vertical dashed lines.

FIG. 7 is a photo of another embodiment of the test kit, including test card 10, packaging 27 for test card 10, sachet of non-alcoholic mouth rinse 14, sample collector 16, cap 18, with built-in dropper tip, funnel 19, and collection swab 20 with sponge head 22.

### DETAILED DESCRIPTION

The present inventor has determined that the oral mucosa and saliva are reservoirs of SARS-CoV-2 virus and its new variants' antigens during infection. Thus, a rapid in vitro immunochromatographic assay for the qualitative detection of SARS-CoV-2 virus and its new variants' antigens in human oropharyngeal lavage has been developed. Methods for collecting a liquid sample (oropharyngeal lavage) from the human mouth and oropharynx and test kits are also disclosed herein. Advantageously, collection of the oropharyngeal lavage is simple, easy, and comfortable, and does not require trained medical personnel to take the sample. Therefore it can be performed in the home by the test subject or family member. A further advantage of the assay is that the virus may be detected earlier than with a NP swab, because the amount of virus can peak in the oral mucosa and saliva before peaking in the nasopharynx.

Oropharyngeal lavage can mean washing out or irrigating the oropharynx by flushing it with a fluid. As used herein, “oropharyngeal lavage” refers to a liquid sample obtained by this process. As such, “oropharyngeal lavage” and “liquid sample” are used interchangeably herein. An abbreviation for oropharyngeal lavage is OPL™ which is a trade mark associated with the presently disclosed methods, assays, and kits.

The oral cavity includes the lips, the inside lining of the lips and cheeks (buccal mucosa), the teeth, the gums, the front two-thirds of the tongue, the floor of the mouth below the tongue, the bony roof of the mouth (hard palate) and the area behind the wisdom teeth (called the retromolar trigone). As used herein, the term “mouth” is synonymous with “oral cavity”. The oropharynx is the middle part of the throat just behind the oral cavity, and defines the oropharyngeal area. It can be seen when your mouth is wide open. It includes the base of the tongue (the back third of the tongue), the soft palate (the back part of the roof of the mouth), the tonsils, and the side and back walls of the throat. The oral cavity and oropharynx help you breathe, talk, eat, chew, and swallow. Minor salivary glands all over the oral cavity and oropharynx make saliva (spit) that keeps your mouth and throat moist, and helps you digest food. The oral mucosa is the mucous membrane lining the inside of the oral cavity and the oropharynx. As mentioned above, the oral mucosa and secreted saliva can harbor viruses such as the SARS CoV-2 virus.

The terms, “non-alcoholic mouth rinse” and “composition” are used interchangeably herein. The non-alcoholic mouth rinse can be obtained by dilution of a stock solution with distilled water.

A method for collecting a liquid sample from the oral cavity and oropharyngeal area comprises avoiding placing food, drink, gum, or tobacco products in the oral cavity for a least 20 minutes prior to collecting the liquid sample; pouring a non-alcoholic mouth rinse into the oral cavity; swishing and gargling the mouth rinse at least once to sweep inside of the oral cavity, including the inside of the cheeks, the tongue, and back of the throat, while not spitting or swallowing the mouth rinse; using a collection swab comprising a sponge head to scrub the inside of the oral cavity, including the inside of the cheeks, the tongue and back of the throat; inserting the collection swab under the tongue; holding the collection swab under the tongue for at least one minute without biting, sucking, or chewing on the sponge head to absorb the mouth rinse; and removing the collection swab from the oral cavity; wherein the sponge head is designed to absorb and discharge the entire volume of the mouth rinse.

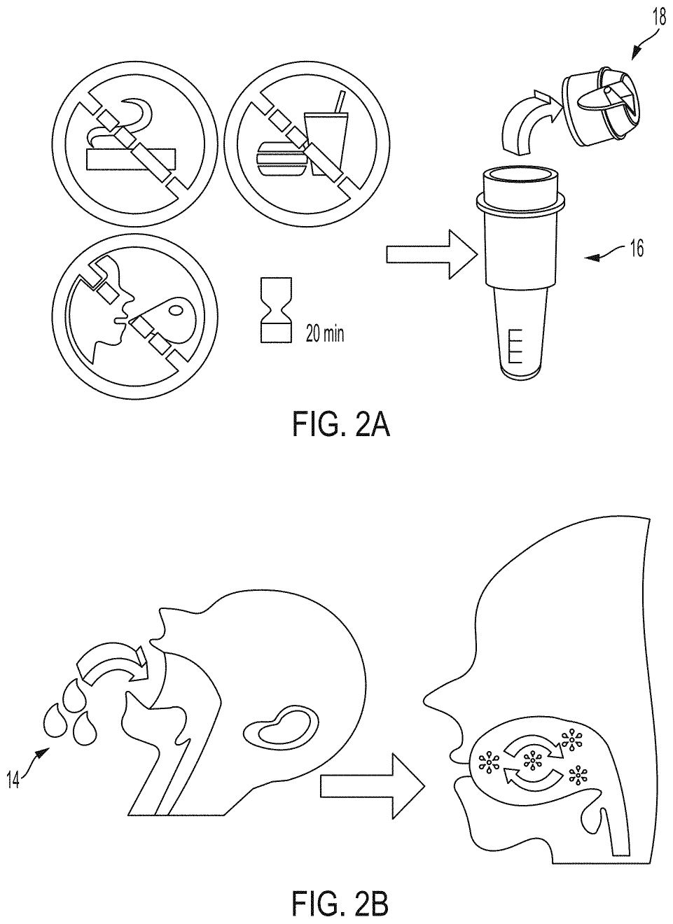

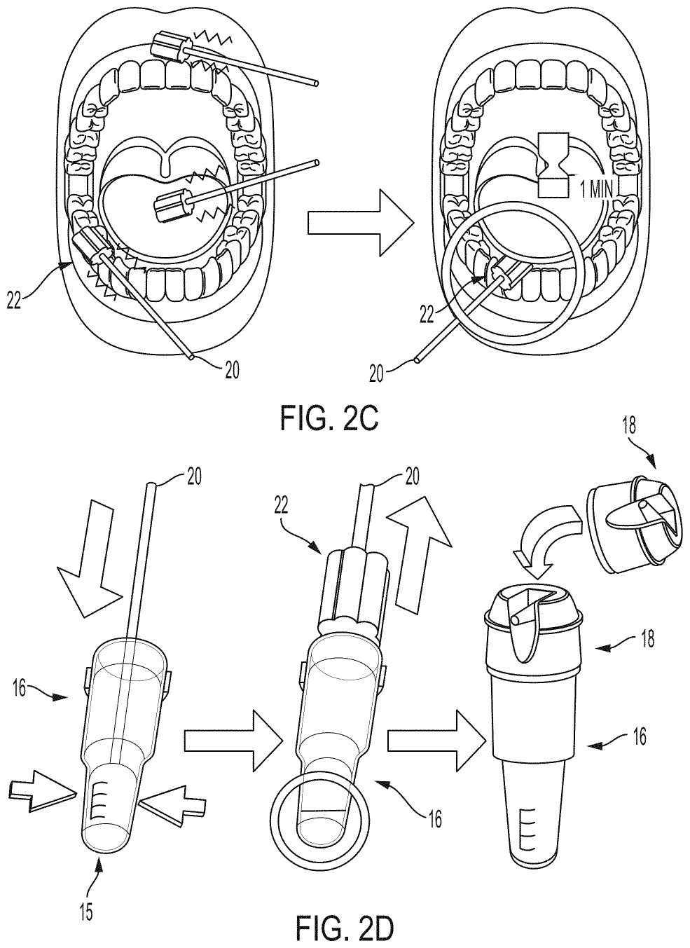

FIG. 2a illustrates schematically no smoking, no eating and drinking, and no chewing gum at least two hours before taking a liquid sample. FIG. 2b illustrates schematically pouring a non-alcoholic mouth rinse 14 into the mouth, swishing and gargling the mouth rinse at least once to sweep inside of the mouth, including the inside of the cheeks, the tongue, and back of the throat, while not spitting or swallowing the mouth rinse.

The non-alcoholic mouth rinse is a composition comprising: a therapeutic antimicrobial mouth rinse; a salt; and a mucolytic agent. Although the composition is characterized as a “non-alcoholic mouth rinse”, its use is not restricted to the oral cavity and oropharyngeal area. It can be generally useful for other medical and diagnostic purposes as well. Thus, “non-alcoholic mouth rinse” and “composition” are used interchangeably herein, and are distinguished from the “therapeutic antimicrobial mouth rinse”, which is a component of the “non-alcoholic mouth rinse”. In some embodiments, the composition is a stock solution comprising, based on the total volume of the composition: 12 to 62% w/v of the therapeutic antimicrobial mouth rinse; 5 to 25% w/v of the salt; and 2 to 8% w/v of the mucolytic agent. The composition can further comprise, based on the total volume of the composition, 15 to 81% w/v of other ingredients, for example, coloring, and flavoring. Thus, in some embodiments, the stock solution further comprises, based on the total volume of the composition, 15 to 81% w/v of at least one of water, a solvent, a flavoring, a sweetener, a coloring, a surfactant, a preservative, or a buffer. The mouth rinse is non-alcoholic, which means that it contains no alcohol, i.e. ethyl alcohol. In some embodiments, the non-alcoholic mouth rinse comprises less than 20, 10, 5, 1, or 0.1% w/v ethyl alcohol.

The stock solution can be diluted with distilled water in a volume ratio of stock solution to distilled water of, for example, 1:2, 1:5, or 1:10, to provide the non-alcoholic mouth rinse for end use in the methods and assays disclosed herein. For example, the non-alcoholic mouth rinse derived from a 1:2 dilution of the stock solution with distilled water can be used for seniors (age about 65 to about 99 years); the non-alcoholic mouth rinse derived from a 1:5 dilution of the stock solution with distilled water can be used for adults (age about 18 to about 64 years); and the non-alcoholic mouth rinse derived from a 1:10 dilution with distilled water can be used for youths (age about 2 to about 17 years). Thus in some embodiments, the non-alcoholic mouth rinse is obtained by diluting the stock solution with distilled water in a volume ratio of stock solution to distilled water of from about 1:2 to about 1:10. The amount of non-alcoholic mouth rinse suitable for use in the methods, assays, and kits disclosed herein can be about 0.5 to about 10 mL, and specifically about 1 to about 5 mL. In some embodiments, the amount of non-alcoholic mouth rinse is about 2 mL for each instance of the method, assay, or kit.

Surprisingly, the composition minimizes false positives in the in vitro immunochromatographic assays utilizing the composition for collecting the liquid sample from the oral cavity and oropharyngeal area. The in vitro immunochromatographic assays for the qualitative detection of SARS-CoV-2 virus antigen utilizing the composition for collecting the liquid sample from the oral cavity and oropharyngeal area provide good agreement with RT-PCR test results, show good analytical sensitivity for different sources of the SARS-CoV-2 virus antigen, provide non-cross reactivity for a variety of bacterial and viral panes, and the test results are not interfered with by various medicinal and endogenous test substances.

The therapeutic antimicrobial mouth rinse component of the composition is an FDA-approved mouth rinse, i.e. a mouth rinse that kills the germs associated with bad breath and gingivitis. Thus, the mouth rinse is effective in disinfecting the sample and the oral cavity. For example, the mouth rinse can be at least 99%, 99.5%, 99.9%, or 99.99% effective in disinfecting the liquid sample. In some embodiments, the mouth rinse is at least 99.99% effective in disinfecting the liquid sample. Thus, in some embodiments of the composition, the therapeutic antimicrobial mouth rinse comprises an antimicrobial that is at least one of cetylpyridinium chloride (CPC), chlorhexidine (CHX), or at least one essential oil selected from the group consisting of eucalyptol, menthol, methyl salicylate, and thymol. In some embodiments of the composition, the therapeutic antimicrobial mouth rinse comprises, based on the total volume of the therapeutic microbial mouth rinse, a combination of 0.092% w/v eucalyptol, 0.042% w/v menthol, 0.060% w/v methyl salicylate, and 0.064% w/v thymol. The concentration of the therapeutic antimicrobial mouth rinse in the stock solution, based on the total volume of the stock solution, can be 12 to 62% w/v. The stock solution can be diluted with distilled water in a volume ratio of stock solution to distilled water of, for example, 1:2, 1:5, or 1:10, to provide the non-alcoholic mouth rinse for end use in the methods and assays disclosed herein.

In some embodiments, the salt can be an alkali metal or alkaline earth metal salt of a halide. Preferably, the salt comprises sodium chloride, to provide a saline solution. The concentration of the salt in the stock solution, based on the total volume of the stock solution, can be 5 to 25% w/v. The stock solution can be diluted with distilled water in a volume ratio of stock solution to distilled water of, for example, 1:2, 1:5, or 1:10, to provide the non-alcoholic mouth rinse for end use in the methods and assays disclosed herein.

In some embodiments of the composition, the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4. The concentration of the mucolytic agent in the stock solution, based on the total volume of the stock solution, can be 2 to 8% w/v. The stock solution can be diluted with distilled water in a volume ratio of stock solution to distilled water of, for example, 1:2, 1:5, or 1:10, to provide the non-alcoholic mouth rinse for end use in the methods and assays disclosed herein.

In some embodiments of the composition, the therapeutic antimicrobial mouth rinse is at least one of cetylpyridinium chloride (CPC), chlorhexidine (CHX), or at least one essential oil selected from the group consisting of eucalyptol, menthol, methyl salicylate, and thymol; the salt comprises sodium chloride; and the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

In some embodiments of the method for collecting a liquid sample from the oral cavity and oropharyngeal area, the rapid in vitro immunochromatographic assay for the qualitative detection of analytes present in human oropharyngeal lavage, the rapid in vitro immunochromatographic assay for the qualitative detection of SARS-CoV-2 virus antigen present in human oropharyngeal lavage, and the kit for rapid in vitro immunochromatographic assay for the qualitative detection of SARS-CoV-2 virus antigen present in human oropharyngeal lavage described herein, the mouth rinse is the composition described above.

FIG. 2c illustrates schematically using a collection swab 20 comprising a sponge head 22 to scrub the inside of the mouth, including the inside of the cheeks, the tongue and back of the throat, and to absorb the mouth rinse; inserting the collection swab 20 under the tongue; and holding the collection swab 20 under the tongue for at least one minute without biting, sucking, or chewing on the collection sponge. The collection swab 20 comprises a sponge head 22 attached to a rod, and can be a dental swab. Dental swabs are well known in the art. The sponge head can have various cross-sectional shapes, e.g. rectangular, square, hexagonal, or round, and the sponge head can also have six or more radial lobes. The sponge head should have sufficient capacity to absorb the entire volume of the mouth rinse used in the collection method.

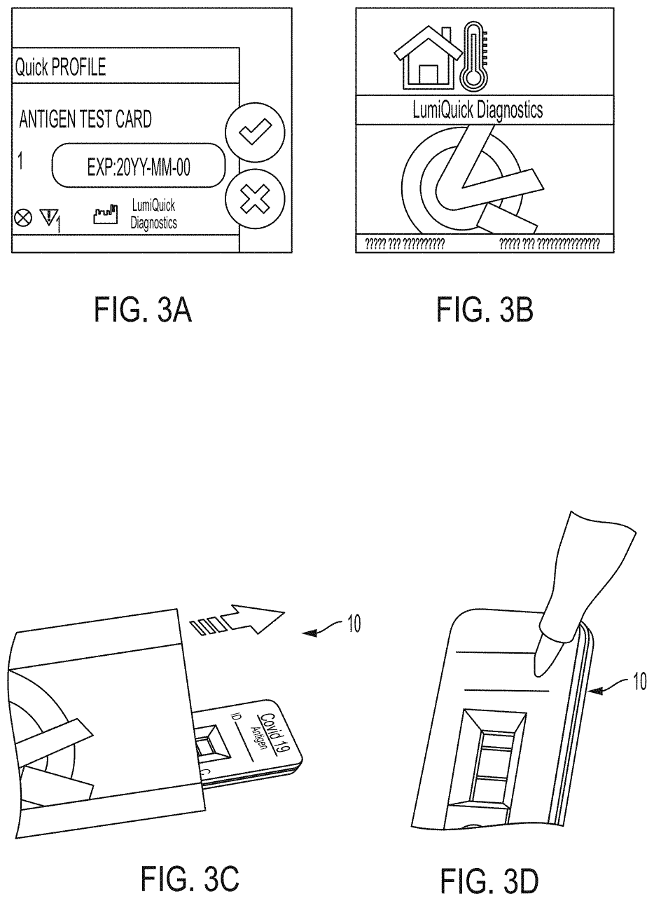

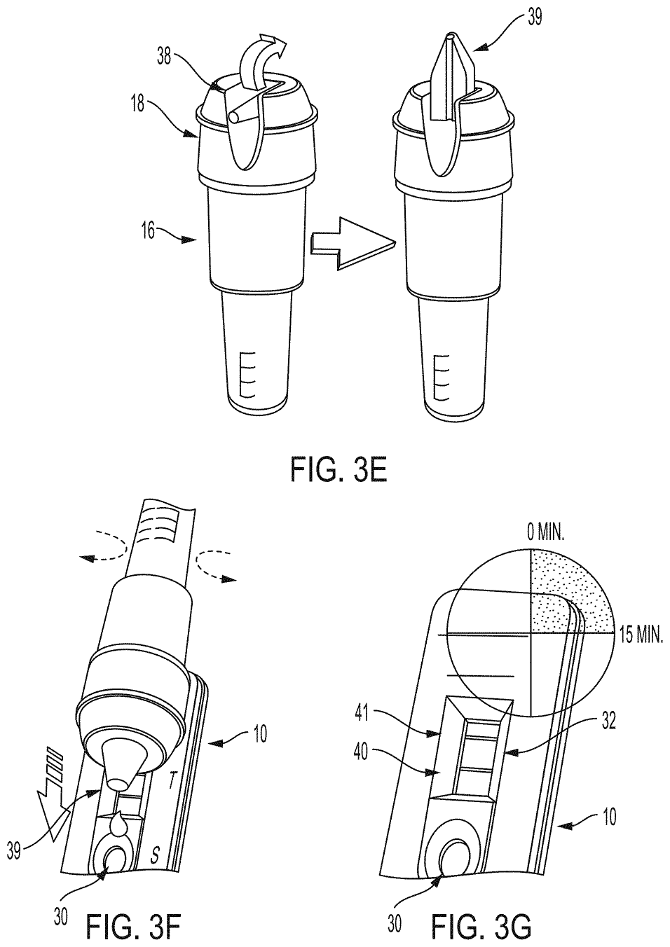

Once the liquid sample is absorbed by the sponge head, it is transferred from the sponge head to the sample collector. Thus, the method further comprises: inserting the collection swab, sponge-end first, into a sample collector wherein at least a section of the sample collector has an inner diameter or width smaller than the width of the sponge head; letting the collection swab inserted into the sample collector stand for at least one minute; pushing the sponge head of the collection swab up and down into the space defined by the inner wall of the section of the sample collector at least three times; pushing the sponge head against an inner wall of the sample collector to squeeze out a maximum amount of liquid sample; and sealing the sample collector with a cap. These steps are illustrated in FIG. 2d. FIG. 2d illustrates inserting the collection swab 20, sponge-end first, into a sample collector 16 wherein at least a section of the sample collector 16 has an inner diameter or width smaller than the width of the sponge head 22; pushing the sponge head 22 of the collection swab 20 up and down into the space defined by the inner wall of the section of the sample collector 16 at least three times; and sealing the sample collector 16 with a cap 18. The cap can comprise a nozzle for discharging drops of the liquid sample. In some embodiments, the nozzle is moveable so as to be open in a first position and closed in a second position. This feature of cap 18 is illustrated in FIG. 3e, which illustrates moving the nozzle in closed position 38 to open position 39.

In some embodiments of the methods disclosed herein, the methods further comprise adding a sample buffer to the liquid sample. The sample buffer can be added as needed.

The collection method is applicable to children as well as adults, with the understanding that infants and toddlers are not expected to gargle and spit out the mouth rinse. Instead of pouring the mouth rinse into the child's mouth, the mouth rinse is absorbed onto the sponge head just prior to use. Thus, a method for collecting a liquid sample from the oral cavity and oropharyngeal area of a human child comprises: avoiding placing food and drink in the oral cavity for a least 20 minutes prior to collecting the liquid sample; absorbing a non-alcoholic mouth rinse into the sponge head of a collection swab; using the collection swab to scrub the inside of the child's oral cavity, including the inside of the cheeks, the tongue and back of the throat; inserting the collection swab under the child's tongue; holding the collection swab under the child's tongue for at least one minute without biting, sucking, or chewing on the sponge head to absorb the mouth rinse; and removing the collection swab from the oral cavity; wherein the sponge head is designed to absorb and discharge the entire volume of the mouth rinse. In some embodiments of this method, the non-alcoholic mouth rinse is a composition comprising: a therapeutic antimicrobial mouth rinse; a salt; and a mucolytic agent. In some embodiments of this method, the composition comprises, based on the total volume of the composition: 12 to 62% w/v of the therapeutic antimicrobial mouth rinse; 5 to 25% w/v of the salt; and 2 to 8% w/v of the mucolytic agent.

With the liquid sample collection method described herein, a rapid in vitro immunochromatographic assay for the qualitative detection of analytes present in human oropharyngeal lavage is possible. The rapid in vitro immunochromatographic assay comprises: collecting a liquid sample from the oral cavity and oropharyngeal area; adding a portion of the liquid sample to the sample port of a test card capable of immunochromatographic assay; and viewing the results on the test card; wherein a first colored band on the test card indicates a positive test result and a second colored band indicates a valid test result. The test card can be a lateral flow test card, in which the direction of the liquid sample in the membrane is horizontal flow. The test card can also be a vertical flow test card, in which the direction of migration of the liquid sample in the membrane is vertical.

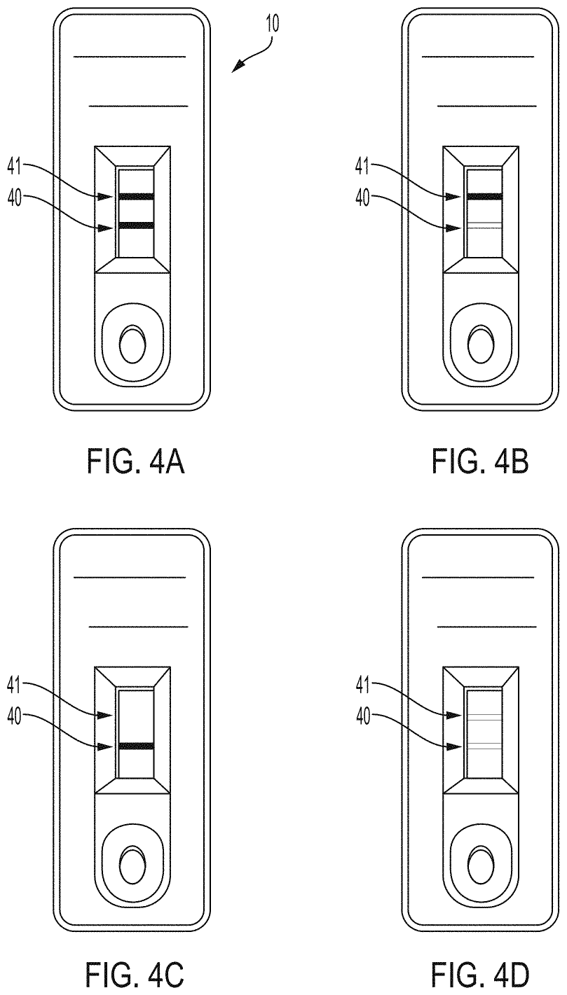

FIG. 4a to 4d illustrate various test card 10 results possible when the test card is designed so that a first colored band indicates a positive test result and a second colored band indicates a valid test result. FIG. 4a illustrates a first colored band 40 indicating a positive test result and a second colored band 41 indicating a valid test result. FIG. 4b illustrates the absence of a first colored band 40 indicating a negative test result and presence of a second colored band 41 indicating a valid test result. FIG. 4c illustrates the presence of a first colored band 40 indicating a positive test result and the absence a second colored band 41 indicating an invalid test result. FIG. 4d illustrates the absence of a first colored band 40 indicating a negative test result and the absence a second colored band 41 indicating an invalid test result.

Analytes that can be tested for in the assay include drugs of abuse and their metabolites. Thus, the analyte can be, for example, 7-acetaminoclonazepam, an alkyl nitrite, alpha-hydroxyalprazolam, alprazolam, 2-amino-2′-chloro-5-nitrobenzophenone, 7-aminoclonazepam, 7-aminonitrazepam, amitriptyline, amobarbital, amoxapine, amphetamine, anabolic steroids, androgen, androstadienone, aprobarbital, atropine, barbiturates, benzodiazepines, benzoylecgonine, benzylpiperazine, boldenone undecylenate, 4-bromo-2,5-dimethoxyphenethylamine, bovine growth hormone, butabarbital, butalbital, butripryline, 4-chlordehydromethyltestosterone, chloroform, clomipramine, clonazepam, clostebol, cocaethylene, cocaine, codeine, codeine-6-glucuronide, cotinine, dehydroepiandrosterone, desipramine, desmethyldiazepam, desoxymethyltestosterone, dexmethylphenidate, dextroamphetamine, dextromethorphan, dextropropoxyphene, dextrorphan, 2,5-diamino-2′-chlorobenzophenone, diamorphine, diazepam, dibenzepin, dihydrotestosterone, dimenhydrinate, 2,5-dimethoxy-4-(n)-propylthiophenethylamine, 2,5-dimethoxy-4-ethylphenethylamine, 2,5-dimethoxy-4-iodophenethylamine, dimethyl ether, dimethyltryptamine, dimethyltryptamine, diphenhydramine hydrochloride, dosulepin hydrochloride, dothiepin hydrochloride, doxepin, drostanolone, ecgonine, ecgonine methyl ester, ephedrine, ergine, estren, 5-estrogen, ethyl-5-(1′-methyl-3′-carboxypropyl)-2-thiobarbituric acid, 5-ethyl-5-(1′-methyl-3′-hydroxybutyl)-2-thiobarbituric acid, ethylestrenol, ethylphenidate, fentanyl, flunitrazepam, fluoxymesterone, furazabol, gamma-hydroxybutyrate, 1-(beta-D-glucopyranosyl) amobarbital, growth hormone, heroine, hexabarbital, human chorionic gonadotropin, human growth hormone, hydrocodone, hydromorphone, (+)-3-hydroxy-N-methylmorphinan, 3-hydroxy clonazepam, 11-hydroxy-tetrahydrocannabinol (11-hydroxy-THC), 3′-hydroxyamobarbital, p-hydroxyamphetamine, p-hydroxynorophedrine, imipramine, iprindole, kava, katamine, levomethylphenidate, iofepramine, lorazepam, lorazepam-glucuronide, lysergic acid diethylamide, meperidine, mescaline, mestanolone, mesterolone, meta-chlorophenylpiperazine, methadone, methamphetamine, methandrostenolone, methcathinone, 3,4-methylenedioxyamphetamine, methanolone, methanolone enanthate, methylenedioxymethamphelamine (ecstacy), methylphenidate, methylphenobarbital, methyl testosterone, mibolerone, (+)-3-morphinan, morphine, nandrolone, nicotine, nitrazepam, N-methyl-diethanciamine, norbolethone, norcodeine, norethandrolone, norketamine, nortriptyline, opiates, opipramol, opium, oxabolone cipionate, oxandrolone, oxazepam, oxycodone, oxymetholone, oxymorphone, pentobarbital, phencyclidine, phenethylamines, phenobarbital, 4-phenyl-4-(1-piperidinyl)-cyclohexanol, 1-phenyl-1-cyclohexene, phenylacetone, 5-[N-(1-phenylcyclohexyl)]-aminopentanoic acid, 1-(1-phenylcyclohexyl)-4-hydroxypiperidine, piperidine, protriptyline, psilocin, psilocybin, quinbolone, salvinorin A, scopolamine, secobarbital, sodium thiopental, stanozolol, telbutal, temazepam, testosterone, testosterone propionate, tetrahydrocannabinol (THC), THC—COOH, tetrahydrogestrinone, toluene, trenbolone, a tricyclic antidepressant, 3-trifluoromethylphenylpiperazine, trimipramine, tryptamines, or any combination thereof. The minimum concentration level at which the presence of any particular drug or metabolite can be detected can be determined by various industry minimum standards, such as those provided, for example, by the National Institute on Drug Abuse (NIDA), the Substance Abuse & Mental Health Services Administration (SAMHSA), and the World Health Organization (WHO).

Analytes that can be tested for in the assay also include infectious agents or the products of infectious agents. The infectious agent or product of an infectious agents can be, for example, Acanthamoeba, aflatoxin, alimentary mycotoxlcoses, altertoxin, amoeba, Anisakis, Ascaris lumbricoides, Bacillus arthracis, Bacillus cereus or its toxin, bacteria, bovine spongiform encephalopathy prioris, Brucella, Caliciviridae, Calymmatobacterium granulomatis, Campylobacter, Campylobacter jejuni, Candida, Candida albicans, Cephalosporium, Chlamydia trachomatis, chronic wasting disease prions, Citrinin, Clostridium botulinum or its toxin, Clostridium perfringens, Corynebacterium ulcerans, Coxielia burnetil, Creutzieldt-Jakob disease prions, Cryptococcus neoformans, Cryptosporidium, Cryptosporidium parvumCycloplazonic acid, Cyclospera cayetanensis, Cytochaiasin, Cytomegalovirus, Diphyilobothrium, Escherichia coli, Ebola, endotoxin, Entamceba histolytica, Enterovirus, Ergopeptine alkaloid, Ergot alkaloid, Ergotamine, Escherichia coli O157, Eustrongylides, Fasciola hepatica, fatal familial insomnia prions, flatworm, Francisella tularensis, Fumitremorgen B.sub.1 Fumonisin, Fusarium, Fusarochromanone, genital warts, Gerstmann-Straussler-Scheinker syndrome prions, Giardia, Giardia lamblia, Granuloma inguinale, H7 enterohemorrhagic, Haemophilus ducreyl, Helicobacter pylori, Hepatitis, Hepatitis A, Hepatitis B, Hepatitis C, Hepatitis D, Hepatitis E, Hepatitis E, herpes simplex virus, Histoplasma capsulatum, HIV, HIV-1, HIV-2, human papillomavirus, influenza, Kaposi's sarcoma-associated herpesvirus, Kojic acid, kuru prions, Listeria monocytogenes, Lolitrem alkaloids, marburg virus, Methicillin-resistant Staphylococcus aureus or its toxin, molluscum, Moniliformin, mononucleosis, mycobacteria, Mycobacterium tuberculosis, Mycoplasma, Mycoplasma hominis, Mycotoxins, Myrothecium, Nanophyetus, Neisseria gonorrhosae, nematode, Nivalenol, Norovirus, Oohratoxins, Oosporeine, parasite, Patulin, Paxilline, Penitrem A, Phomopsins, Plasmodium, Platyhelminthes, Plesiomonas shigelloides, Pneumococcus, Pneumocystis jirovecii, prions, protozoa, rhinovirus, Rotavirus, Salmonella, Sarcocystis hominis, Sarcocystis sulhominis, scraple prions, sexually transmitted disease, Shigella, Shigella, Sporidesmin A, Stachybotrys, Staphylococcus aureus or its toxin, Sterigmatocystin, Streptococcus, Streptococcus pneumoniae, Streptococcus pyogenes, Taenia saginata, Taenia solium, tapeworm, Tenia solium, Tinea, Toxoplasma gondii, Tremorgenic mycotoxins, Treponema palidum, Trichinella spiralis, Trichoderma, Trichomonas vaginalis, Trichothecene, Trichuris trichlura, Typanosoma cruzi, Ureaplasma urealyticum, Verrucosidin, Varruculogen, Vibrio cholerae non-O1, Vibrio cholerae O1, Vibrio-parahaemolyticus, Vibrio vulnificus, viruses, yeast infections, Yersinia enterocolitica, Yersinia pseudotuberculosis, Zearalenois, Zearalenone, antibodies against any of the foregoing, or any combination thereof.

Analytes that can be tested for in the assay also include allergens. The allergen can be, for example, aesculus, aider, almonds, animal products, arternisia vulgaris, beans, bet sting venom, birch, calyx, cat dander, celeriac, celery, Chenopodium album, cockroach, corn, dander, dong dander, drugs, dust mite excretion, egg albumen, eggs, Fel d 1 protein, fruit, fur, grass, hazel, hornbeam, insect stings, latex, legumes, local anaesthetics, maize, metal, milk, mold spores, mosquito saliva, mouse dander, nettle, olea, peanuts, peas, pecans, penicillin, Plant pollens, plantago, platanus, poplar, pumpkin, ragweed, rat dander, ryegrass, salicylates, seafood, sesame, sorrel, soy, soybeans, sulfonamides, tilia, timothy-grass, tree nuts, trees, wasp sting venom, weeds, wheat, willow, antibodies against any of the foregoing, or any combination thereof.

Analytes that can be tested for in the assay also include pollutants, toxins, and contaminants. The pollutant, toxin, or contaminant can be, for example, 1,2-dibromoethane, acrylamide, aldehydes, arsenic, artificial growth hormone, asbestos, benzene, benzopyrene, carcinogens, dichloro-diphenyl-trichloroethane, formaldehyde, kepone, lead, mercury, methylmercury, nitrosamines, N-nitroso-N-methylurea, organochlorine insecticides, pesticides, polychlorinated biphenyls, polychlorinated dibenzofurans, polychlorinated dibenzo-p-dioxins, recombinant bovine growth hormone, recombinant bovine somatotropin, toluene, vinyl chloride, antibodies against any of the foregoing, or any combination thereof.

Analytes that can be tested for in the assay also include analytes with diagnostic or medical value. The analyte with diagnostic or medical value can be, for example, acid phosphatase, active-B12, AFP, Alanine Aminotransferase, Alanine Aminotransferase, Albumin, Albumin BCG, Albumin BCP, Alkaline Phosphatase, Alpha-1 Antitrypsin, Alpha-1 Glycoprotein, Amikacin, Ammonia, Amylase, Anti-CCP, Anti-Tg, Anti-TPO, Apolipoprotein A1, Apolipoprotein B, ASO, Asparate Aminotransferase, Aspartate Aminotransferase, B12, Beta2 Microglobulin, Beta2 Microglobulin, BNP, CA 125, CA 125 II, CA 15-3, CA 19-9 XR, Calcium, Carbamazepine, Carbon Dioxide, CEA, Ceruloplasmin, Cholesterol, CK-MB, Complement C3, Complement C4, Cortisol, C-Peptide, C-Reactive Protein, Creatine Kinase, Creatinine, CRP Vario, Cyclosporine, Cyclosporine and Metabolite-Whole Blood, Cyclosporine Monoclonal-Whole Blood, D-Dimer, DHEA-S, Digitoxin, Digoxin, Digoxin, Digoxin II, Digoxin III, Direct Bilirubin, Direct LDL, Estradiol, Ferritin, FLM II, Folate, Free Carbamazepine, Free Phenytoin, Free PSA, Free T3, Free T4, Free Valproic acid, FSH, Gamma-Glutamyl Transferase, Gentamicin, Glucose, Glycated Hemoglobin, Haptoglobin, hCG, Hemoglobin, Homocysteine, ICT CI-, IGFBP-1, Immunoglobulin, Immunoglobulin A, Immunoglobulin E, immunoglobulin G, Immunoglobulin M, Insulin, Intact PTH, Iron, K+, Kappa Light Chain, Lactate Dehyrogenase, Lactic acid, Lambda Light Chain, LH, Lidocaine, Lipase, Lithium, Lp, magnesium, metabolites, Methotrexate II, Microalbumin, MPO, Myoglobin, Na+, N-Acetyl-procainamide, neonatal Bilirubin, NGAL, P-Amylase, Pepsinogen I, Pepsinogen II, Phenobarbital, Phenytoin, Phosphorus, Prealbumin Procainamide, Progesterone, Prolactin, Quinidine, Rheumatoid Factor, SHBG, Sirollmus, STAT CK-MB, T4, Tacrolimus, Tacrolimus II, Testosterone, Tg, Theophylline, Theophylline II, TIBC, TIMP-1, Tobramycin, Total Bi, lirubin, Total Estriol, Total Protein, Total PSA, Total T3, Total T4, Transferrin, Triglycerides, Troponin-I, Troponin-I ADV, TSH, T-Uptake, UIBC, Ultra HDL, Urea Nitrogen, Uric Acid, Urine/CSF Protein, Valproic Acid, Vancomycin, Vancomycin II, Vitamin D, antibodies against any of the foregoing, or any combination thereof.

Adding a portion of the liquid sample to the sample port of a test card capable of lateral flow immunochromatographic assay is illustrated in FIG. 3f, in which the liquid sample is added to sample port 30 of test card 10. Sample port 30 is labelled “S” directly on test card 10. Three drops is a suitable portion of the liquid sample to add to the sample port, although more or less drops can also be used. As shown schematically in FIG. 3f, the adding can be done by inverting the sample collector 16 and squeezing the end opposite the cap nozzle. In some embodiments, cap 18 comprises a nozzle for discharging the liquid sample in which the nozzle can be moved from a closed position to an open position. FIG. 3e illustrates moving the nozzle in closed position 38 to open position 39.

The in vitro immunochromatographic assay also includes viewing the results on the test card; wherein a first colored band on the test card indicates a positive test result and a second colored band indicates a valid test result. This is illustrated in FIG. 3g where first colored band 40 of test card 10 indicates a positive test result and second colored band 41 indicates a valid test result. An internal reagent added to the test card membrane provides the second colored band, which is a procedural control band. Good laboratory practice prescribes daily testing of externally applied control samples to validate the reliability of the test card. As indicated schematically in FIG. 3g, the first and second colored bands should appear within 15 minutes after applying the liquid sample to the test card. However, more, or less, time may be required for the appearance of the colored bands.

The collection of liquid sample (oropharyngeal lavage) step in the in vitro immunochromatographic assay can be the method for collecting a liquid sample from the human mouth and oropharynx discussed above. Thus, in some embodiments of the in vitro immunochromatographic assay, the collecting comprises: avoiding placing food, drink, gum, or tobacco products in the mouth for a least 20 minutes prior to collecting the liquid sample; pouring a non-alcoholic mouth rinse into the oral cavity; swishing and gargling the mouth rinse at least once to sweep inside of the mouth, including the inside of the cheeks, the tongue, and back of the throat, while not spitting or swallowing the mouth rinse; using a collection swab comprising a sponge head to scrub the inside of the oral cavity, including the inside of the cheeks, the tongue and back of the throat, and to absorb the mouth rinse; inserting the collection swab under the tongue; holding the collection swab under the tongue for at least one minute without biting, sucking, or chewing on the sponge head to absorb the mouth rinse; and removing the collection swab from the oral cavity; wherein the sponge head is designed to absorb and discharge the entire volume of the mouth rinse. In some embodiments of this in vitro immunochromatographic assay, the non-alcoholic mouth rinse is a composition comprising: a therapeutic antimicrobial mouth rinse; a salt; and a mucolytic agent. In some embodiments of this in vitro immunochromatographic assay, the composition comprises, based on the total volume of the composition: 12 to 62% w/v of the therapeutic antimicrobial mouth rinse; 5 to 25% w/v of the salt; and 2 to 8% w/v of the mucolytic agent.

The collection of liquid sample (oropharyngeal lavage) step in the in vitro immunochromatographic assay further comprises transferring the liquid sample from the sponge head to the sample collector as in the method for collecting a liquid sample from the human mouth and oropharynx discussed above. Thus, in some embodiments of the in vitro immunochromatographic assay, the collecting further comprises: inserting the collection swab, sponge-end first, into a sample collector wherein at least a section of the sample collector has an inner diameter or width smaller than the width of the sponge head; letting the collection swab inserted into the sample collector stand for at least one minute; pushing the sponge head of the collection swab up and down into the space defined by the inner wall of the section of the sample collector at least three times; pushing the sponge head against an inner wall of the sample collector to squeeze out a maximum amount of liquid sample; and sealing the sample collector with a cap. As in the method for collecting a liquid sample, the cap can comprise a nozzle for discharging drops of the liquid sample. In some embodiments, the nozzle is moveable so as to be open in a first position and closed in a second position. This feature of cap 18 is illustrated in FIG. 3e, which illustrates moving the nozzle in closed position 38 to open position 39.

The in vitro immunochromatographic assay can further comprise additional steps, e.g. checking the test card expiration date, bringing the kit components to room temperature, removing test card from its packaging, and labelling the test card with a test identification number (ID), as illustrated schematically in FIGS. 3a, 3b, 3c, and 3d, respectively.

Oropharyngeal lavage and the method for collecting oropharyngeal lavage from the human mouth and oropharynx disclosed herein are useful for in vitro immunochromatographic assay for the qualitative detection of SARS-CoV-2. Thus, a rapid in vitro immunochromatographic assay for the qualitative detection of SARS-CoV-2 virus antigen present in human oropharyngeal lavage, comprises collecting a liquid sample from the oral cavity and oropharyngeal area; adding a portion of the liquid sample to the sample port of a test card capable of detecting the SARS-CoV-2 virus antigen; and viewing test results on the test card, wherein a first colored band on the test card indicates a positive test result and a second colored control band indicates a valid test result. The test card can be a lateral flow test card, in which the direction of the liquid sample in the membrane is horizontal flow. The test card can also be a vertical flow test card, in which the direction of migration of the liquid sample in the membrane is vertical. In some embodiments of this in vitro immunochromatographic assay, the non-alcoholic mouth rinse is a composition comprising: a therapeutic antimicrobial mouth rinse; a salt; and a mucolytic agent. In some embodiments of this in vitro immunochromatographic assay, the composition comprises, based on the total volume of the composition: 12 to 62% w/v of the therapeutic antimicrobial mouth rinse; 5 to 25% w/v of the salt; and 2 to 8% w/v of the mucolytic agent.

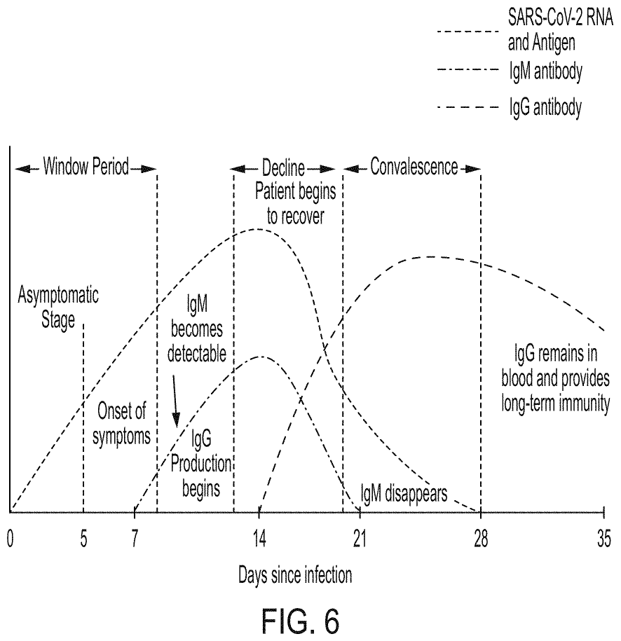

FIG. 6 is a schematic plot of SARS-CoV-2 RNA and antigen, IgM antibody, and IgG antibody levels as a function of days since infection. FIG. 6 also indicates the asymptomatic stage (0 to 5 days), the onset of symptoms (ca. 5 to 8 days), decline (recovery) stage (ca. 13 to 20 days), and convalescent stage (ca. 20 to 28 days), all delineated by vertical dashed lines. Although levels of RNA, antigen, and IgM antibody peak at ca. day 14, i.e. 9 days after the onset of symptoms, COVID-19 antigen tests in general have the best sensitivity from day 1 to day 5 after the onset of symptoms according to CDC guidance. Antigen levels in specimens collected beyond day 5 to day 7 after the onset of symptoms may drop below the limit of detection of the test.

Although, antigen levels in samples collected beyond 5 to 7 days of the onset of symptoms may drop below the limit of detection of the assay, the early detection capability of the assay disclosed herein provides an advantage in early management of COVID-19 infections over antibody tests. For example, earlier detection of an infection allows for earlier following of quarantine protocols, thereby avoiding unintentional infection of loved ones and friends one is normally in frequent contact with, as well as avoiding spreading the virus to the general population. FIG. 6 further shows that IgG antibody production does not begin until day 14 after infection and does not peak until ca. day 25 after infection. Therefore IgG antibody tests are not useful for rapid detection of infection.

The collection of liquid sample (oropharyngeal lavage) step in the in vitro immunochromatographic assay for the qualitative detection of SARS-CoV-2 virus antigen can be the same method for collecting a liquid sample from the human mouth and oropharynx discussed above. Thus, in some embodiments of the in vitro immunochromatographic assay for the qualitative detection of SARS-CoV-2 virus antigen, the collecting comprises: avoiding placing food, drink, gum, or tobacco products in the oral cavity for a least 20 minutes prior to collecting the liquid sample; pouring a non-alcoholic mouth rinse into the oral cavity; swishing and gargling the mouth rinse at least once to sweep inside of the oral cavity, including the inside of the cheeks, the tongue, and back of the throat, while not spitting or swallowing the mouth rinse; using a collection swab comprising a sponge head to scrub the inside of the oral cavity, including the inside of the cheeks, the tongue and back of the throat; inserting the collection swab under the tongue; holding the collection swab under the tongue for at least one minute without biting, sucking, or chewing on the sponge head to absorb the mouth rinse; and removing the collection swab from the oral cavity; wherein the sponge head is designed to absorb and discharge the entire volume of the mouth rinse.

The collection of liquid sample (oropharyngeal lavage) step in the in vitro immunochromatographic assay for the qualitative detection of SARS-CoV-2 virus antigen, the collecting further comprises transferring the liquid sample from the sponge head to the sample collector as in the method for collecting a liquid sample from the human oral cavity and oropharynx discussed above. Thus, in some embodiments of the in vitro immunochromatographic assay for the qualitative detection of SARS-CoV-2 virus antigen, the collecting comprises: inserting the collection swab, sponge-end first, into a sample collector wherein at least a section of the sample collector has an inner diameter or width smaller than the width of the sponge head; letting the collection swab inserted into the sample collector stand for at least one minute; pushing the sponge head of the collection swab up and down into the space defined by the inner wall of the section of the sample collector at least three times; pushing the sponge head against an inner wall of the sample collector to squeeze out a maximum amount of liquid sample; and sealing the sample collector with a cap. As in the method for collecting a liquid sample, the cap can comprise a nozzle for discharging drops of the liquid sample. In some embodiments, the nozzle is moveable so as to be open in a first position and closed in a second position. This feature of cap 18 is illustrated in FIG. 3e, which illustrates moving the nozzle in closed position 38 to open position 39.

The test card utilizes mouse monoclonal antibodies to detect the Nucleocapsid protein of SARS-CoV-2 virus in oropharyngeal lavage (liquid samples). The anti-SARS CoV-2 antibodies are coated onto the membrane providing a capture zone and conjugated to colloidal gold as the detection probe. The membrane can be composed of nitrocellulose. When a liquid sample is applied to a sample port of the test card, and the liquid sample contains SARS-CoV-2 viral antigens, the antigens will form an antigen-antibody complex with the anti-SARS CoV-2 gold conjugate. The complex will continue to move through the membrane by capillary action to be captured by anti-SARS CoV-2 antibodies coated on a test zone to form a colored band indicating a positive result. Absence of a colored band on the test zone indicates a negative result. A built-in colored control band will always appear when the test is performed properly, whether the SARS-CoV-2 virus is present or not. Thus, in some embodiments, the in vitro immunochromatographic assay for the qualitative detection of SARS-CoV-2 virus antigen present in human oropharyngeal lavage, the test card comprises: a membrane; anti-SARS CoV-2 antibody-detection probe conjugates coated onto a capture zone of the membrane; anti-SARS CoV-2 antibodies coated onto a test zone of the membrane; a sample port; and a viewing port; wherein when the liquid sample contains SARS-CoV-2 viral antigens, the antigens form an antigen-antibody complex with the anti-SARS-CoV-2-detection probe conjugate, and the antigen-antibody complex is captured by anti-SARS CoV-2 antibodies coated onto the test zone to form a first colored band indicating a positive result; and wherein a second colored control band appears when the result is valid.

The components needed to do the rapid in vitro immunochromatographic assay for the qualitative detection of SARS-CoV-2 virus antigen present in human oropharyngeal lavage can be contained in a kit. Thus, a kit for rapid in vitro immunochromatographic assay for the qualitative detection of SARS-CoV-2 virus antigen present in human oropharyngeal lavage comprises: a collection swab comprising a sponge head; a non-alcoholic mouth rinse; a sample collector; a cap for the sample collector; a test card capable of detecting the SARS-CoV-2 virus antigen; and instructions for conducting the assay. The cap can comprise a nozzle for discharging drops of the liquid sample. In some embodiments, the cap comprises a nozzle for discharging the liquid sample in which the nozzle can be moved from a closed position to an open position. This feature of cap 18 is illustrated in FIG. 3e, which illustrates moving the nozzle in closed position 38 to open position 39. In some embodiments of this kit, the non-alcoholic mouth rinse is a composition comprising: a therapeutic antimicrobial mouth rinse; a salt; and a mucolytic agent. In some embodiments of this kit, the composition comprises, based on the total volume of the composition: 12 to 62% w/v of the therapeutic antimicrobial mouth rinse; 5 to 25% w/v of the salt; and 2 to 8% w/v of the mucolytic agent.

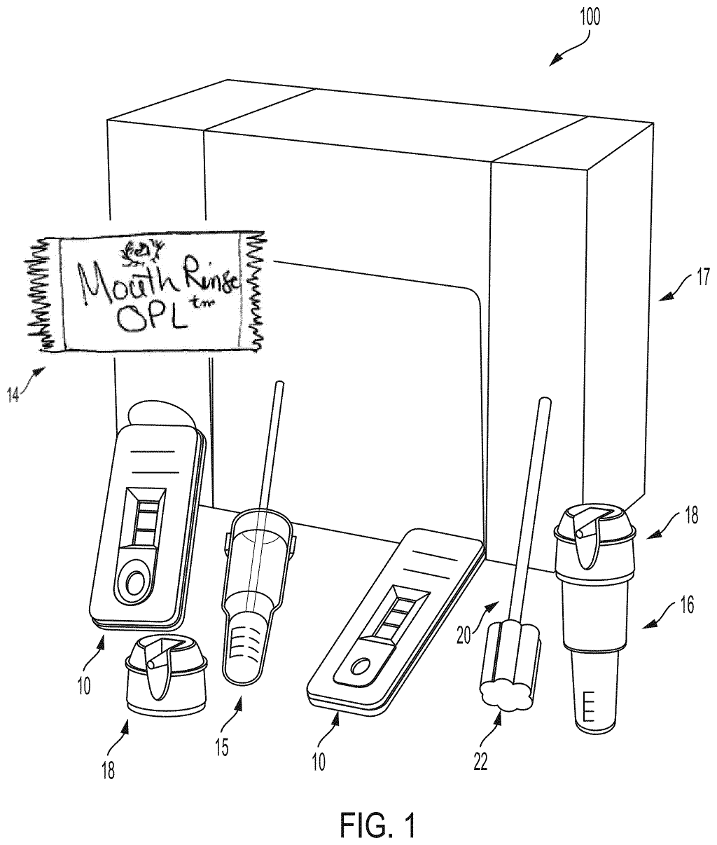

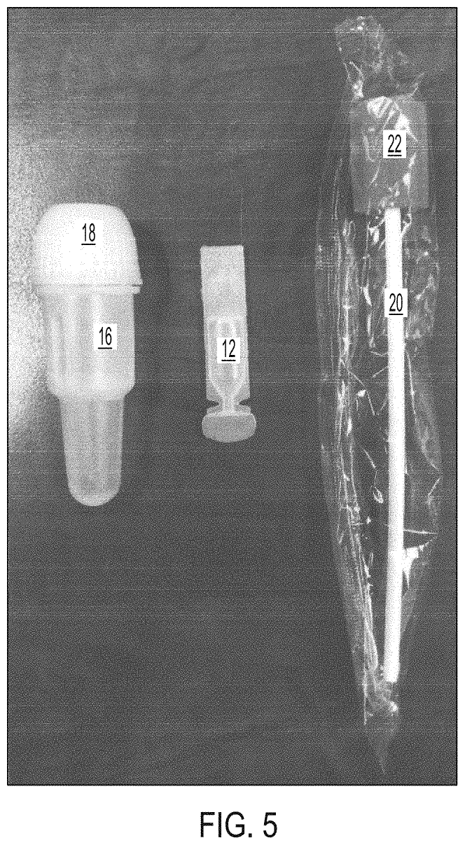

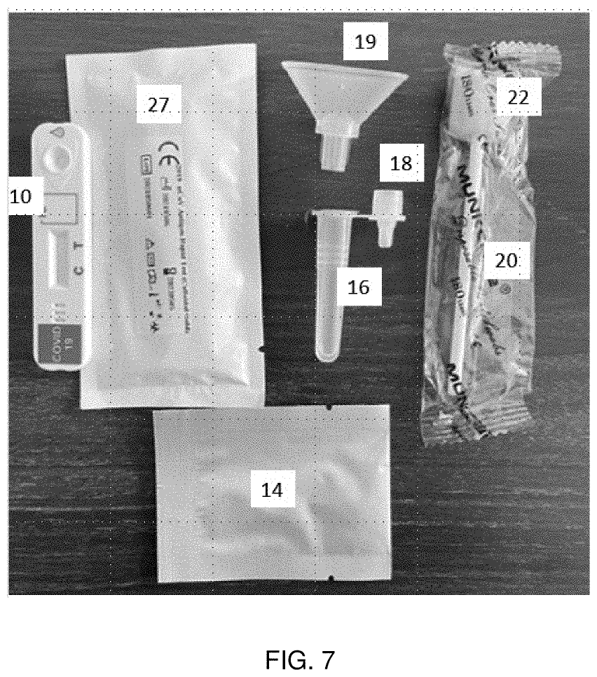

An embodiment of the kit is depicted schematically in FIG. 1. Kit 100 comprises a collection swab 20 with sponge head 22, sample collector 16 with cap 18 attached, test card 10 capable of detecting the SARS-CoV-2 virus antigen, and a sachet of non-alcoholic mouth rinse 14. In this embodiment, the sachet contains 2 mL of non-alcoholic mouth rinse. Packaging 17, 15 (collection swab 20 inserted into sample collector 16 as intended), a separate collector cap 18, and a second test card 10 are also shown. This embodiment of the kit is commercially available as the “Quick PROFILE™ COVID-19 Antigen Test”, and is also known as the “Quick PROFILE™ COVID-19 OPL™ Antigen Test”. FIG. 5 is a photo of selected components of another embodiment of the kit, i.e. sample collector 16 with cap 18 attached, sample buffer 12, and collection swab 20 with sponge head 22. The kit can comprise components sufficient to conduct any practical number of immunochromatographic assays. In some embodiments, the kit can comprise enough components to conduct up to ten immunochromatographic assays. The kit can comprise an equal number of test cards, sample collectors, caps, and collection swabs. In some embodiments, the kit comprises up to ten each of test cards, sample collectors, caps, and collection swabs. FIG. 7 is a photo of another embodiment of the test kit, including test card 10, packaging 27 for test card 10, sachet of non-alcoholic mouth rinse 14, sample collector 16, cap 18, with built-in dropper tip, funnel 19, and collection swab 20 with sponge head 22.

In some embodiments, the test card in the kit comprises: a membrane; anti-SARS CoV-2 antibody-detection probe conjugates coated onto a capture zone of the membrane; anti-SARS CoV-2 antibodies coated onto a test zone of the membrane; a sample port; and a viewing port; wherein when the liquid sample contains SARS-CoV-2 viral antigens, the antigens form an antigen-antibody complex with the anti-SARS-CoV-2-detection probe conjugate, and the antigen-antibody complex is captured by anti-SARS CoV-2 antibodies coated onto the test zone to form a first colored band indicating a positive result; and wherein a second colored control band appears when the result is valid. The test card can be a lateral flow test card, in which the direction of the liquid sample in the membrane is horizontal flow. The test card can also be a vertical flow test card, in which the direction of migration of the liquid sample in the membrane is vertical.

Certain precautions are recommended when using the kit for the vitro immunochromatographic assay for the qualitative detection of SARS-CoV-2 virus antigen present in human oropharyngeal lavage. The kit should not be used beyond the expiration date on the outside of the box. The test card should be stored at 4 to 30° C. sealed in its original protective foil pouch, and never frozen. The expiration date is based on the assumption of storage at 4 to 30° C. Different lots of components of the kit should not be interchanged or mixed. The test card should not be inserted directly into the oral cavity or oropharyngeal area. Any test results obtained after 20 minutes after applying the liquid sample to the sample port should be disregarded. Appropriate precautions for the collection, handling, storage, and disposal of liquid samples should be taken. Personal protective equipment (PPE) should be used when handling liquid samples. Containers and used contents, including liquid samples, should be disposed of in compliance with relevant Federal, state, and local regulations. Kit components should not be reused. The test card must remain sealed in its protective foil pouch until use. Inadequate or inappropriate liquid sample collection, storage, and transport may result in inaccurate test results. If infection with the SARS-CoV-2 virus is suspected based on current clinical and epidemiological screening criteria recommend by public health authorities, liquid samples should be collected with appropriate infection control precautions and sent to state or local health departments for further testing. Viral cultures should not be attempted unless a BSL 3+ facility is available to receive and culture the liquid samples.

There are certain limitations that should be understood with the rapid in vitro immunochromatographic assay for the qualitative detection of SARS-CoV-2 virus antigen present in human oropharyngeal lavage. Failure to follow the test procedure disclosed herein and in the instructions for conducting the assay provided with the kit may adversely affect test performance and/or invalidate the test result. A negative test result may occur if the level of antigen in a liquid sample is below the limit of detection. A negative test result does not rule out other potential non-SARS CoV-2 viral infections. A negative test result also does not rule out co-infection with other pathogens. Moreover, the monoclonal anti-SARS CoV-2 antibodies may fail to detect, or may detect with less sensitivity, SARS-CoV-2 viruses that have undergone minor amino acid changes in the target epitope region. In view of these potential issues, negative test results should be confirmed by molecular diagnosis if a COVID-19 infection is suspected. Moreover, the test results, positive or negative, should always be evaluated in conjunction with other clinical data available to the patient's physician.

The method for collecting a liquid sample from the oral cavity and oropharyngeal area disclosed herein make analyte detection, and in particular, detection of SARS CoV-2 virus antigen, easier to perform and more accessible to non-trained people outside the medical profession. Point-of-care testing (POCT or bedside testing) refers to medical diagnostic testing at or near the point of care, that is, at the time and place of patient care. This contrasts with the historical pattern in which testing was wholly or mostly confined to medical laboratories, which entailed sending off specimens away from the point of care and then waiting hours or days to learn the results. During this time, care must continue without the needed diagnostic information. Advantageously, the rapid in vitro immunochromatographic assay for the qualitative detection of analytes present in human oropharyngeal lavage disclosed herein is ideally suited for point-of-care use, and even home use. In particular, the rapid in vitro immunochromatographic assay for the qualitative detection of SARS-CoV-2 virus antigen present in human oropharyngeal lavage disclosed herein is an aid in the rapid diagnosis of SARS-CoV-2 virus infections. Advantageously, it is easy to collect the liquid sample, and the assay is easy to conduct and results are rapidly obtained.

In particular, the method for collecting a liquid sample from the oral cavity and oropharyngeal area disclosed herein is an improvement over prior art nasopharyngeal (NP) sampling. NP sampling is most effective when done by professionals. Although nasal mid-turbinate swabs are somewhat less intrusive than NP swabs, both can cause irritation and sneezing, which can cause spread of SARS-CoV-2. Moreover, nasal swabs can miss patients with low SARS-CoV-2 viral loads. (Callahan et al. medRxiv preprint doi: https://doi.org/10.1101/2020.06.12.20128736) Finally, viral shedding during the course of infection is altered for Omicron, with higher viral shedding in saliva compared to nasal samples. (Marais et al., medRxiv preprint doi: https://doi.org/10.1101/2021.12.22.21268246) In view of the above, the method for collecting a liquid sample from the oral cavity and oropharyngeal area disclosed herein is an improvement over nasal sampling. Advantageously, it can be performed in the home by the test subject or family member, it does not cause irritation or sneezing, and it takes advantage of the higher viral shedding of Omicron in saliva relative to nasal samples. Moreover, the mouth rinse used in the method disinfects both the liquid sample and the oral cavity, which helps to reduce the spread of the virus.

The following examples are provided as representative. These examples are not to be construed as limiting the scope of the present embodiments or other equivalent embodiments that will be apparent to the skilled person in the art in view of this disclosure, drawings, and appended claims.

### EXAMPLES

### Example 1. Clinical Evaluation

Clinical evaluation of the rapid in vitro immunochromatographic assay for the qualitative detection of SARS-CoV-2 virus antigen present in human oropharyngeal lavage was conducted in different countries. The number of positive and number test results were compared to the number of positive and negative test results obtained with the same liquid samples by RT-PCR, which refers to reverse transcription polymerase chain reaction. The results are summarized in Table 1 below. As can be seen from Table 1, the immunochromatographic assay results tracked very closely with the RT-PCR test results, with only two of the RT-PCR positive samples testing negative in the immunochromatographic assay.

TABLE 1
Clinical Evaluation, Numbers of Test Results

| | RT-PCR Positive | RT-PCR Negative |
|---|---|---|
| | 54 | 445 |
| Immunochromatographic | 52 | 2 |
| assay - Positive | | |
| Immunochromatographic | 0 | 445 |
| assay - Negative | | |

### Example 2. Analytical Sensitivity

The limit of detection (LOD) for the rapid in vitro immunochromatographic assay for the qualitative detection of SARS-CoV-2 virus antigen was established in an analytical sensitivity study preformed with three virus strains and three recombinant nucleocapsid proteins. The LOD was defined as the analytical concentration corresponding to a 95% positive rate. A total of ten replicates at the target concentration of each analyte listed in Table 2 showed 100% positive results. TCID₅₀ refers to a fifty-percent tissue culture infective dose

TABLE 2
Analytical Sensitivity

| Analyte | Limit of Detection |
|---|---|
| SARS-CoV-2, USA-WA 1/2020 | 3.80 × 10² TCID₅₀/mL |
| SARS-CoV-2, HK/VM20001061/2021 | 3.16 × 10² TCID₅₀/mL |
| SARS-CoV-2, Italy-INMI1 | 9.55 × 10² TCID₅₀/mL |
| Recombinant N Protein 1 | <1 ng/mL |
| Recombinant N Protein 2 | <1 ng/mL |
| Recombinant N Protein 3 | <1 ng/mL |

### Example 3. Cross Reactivity

The cross reactivity of the rapid in vitro immunochromatographic assay for the qualitative detection of SARS-CoV-2 virus antigen was evaluated with six bacteria and 18 viruses. The results are summarized in Table 3 below. None of the microorganisms tested gave a positive test result at the reported concentration. Therefore, the specificity of non-cross reactivity is 100%. CFU refers to colony-forming unit.

TABLE 3
Cross Reactivity

| Bacterial Panel | Test Concentration (CFU/mL) |
|---|---|
| Escherichia coli, clinical isolate | 7.92 × 10⁸ |
| Haemophilus influenzae, Type B, Egypt | 5.43 × 10⁷ |
| Pseudomonas aeruginosa, clinical isolate | 8.44 × 10⁸ |
| Staphylococcus aureus, MRSA, COL | 1.84 × 10⁸ |
| Staphylococcus epidermidis, MRSE, RP62A | 9.27 × 10⁸ |
| Staphylococcus pneumoniae, Z022 19F | 4.16 × 10⁵ |

| Viral Panel | Test Concentration (TCID₅₀/mL) |
|---|---|
| Corona virus (HCoV-OC43) | 1.65 × 10⁵ |
| Corona virus (HCoV-NL63) | 1.41 × 10⁴ |
| Corona virus (HCoV-229E) | 4.17 × 10⁴ |
| Rhinovirus A2 | 3.39 × 10³ |
| Influenza A virus H1N1 Brisbane/59/07 | 7.24 × 10⁴ |
| Influenza A virus H3N2 Brisbane/10/07 | 4.17 × 10⁴ |
| Influenza B virus H1N1 Florida/02/06 | 1.26 × 10⁵ |
| Parainfluenza virus Type 1 | 5.01 × 10⁴ |
| Parainfluenza virus Type 2 | 1.05 × 10⁵ |
| Parainfluenza virus Type 3 | 8.51 × 10⁷ |
| Parainfluenza virus Type 4A | 1.51 × 10⁵ |
| Human Metapneumovirus 16 Type Al | 1.26 × 10⁵ |
| Adeno virus Type 4 | 5.01 × 10⁵ |
| Respiratory syncytial virus Type A | 1.26 × 10⁵ |
| Respiratory syncytial virus Type B | 1.26 × 10⁵ |
| Enterovirus Type 68 | 3.80 × 10⁵ |
| Enterovirus Type 71 | 1.65 × 10⁵ |
| MERS-CoV virus Florida/USA-2 Saudi Arabia | 3.55 × 10⁴ |
| 2014 | |

### Example 4. Interference

Exogenous substances, such as medications, and endogenous substances were evaluated for interference with the rapid in vitro immunochromatographic assay for the qualitative detection of SARS-CoV-2 virus antigen by spiking the substance into the sample buffer with or without 1×LOD of SARS-CoV-2 virus and tested with six replicates. The results are summarized in Table 4 below. The results were 100% positive for samples spiked with the virus and 100% negative for samples without the virus. Therefore, none of the tested substances interfered with the assay at the reported concentrations.

TABLE 4
Interference Substances and Test Concentrations

| Interference Substance | Test Concentration |
|---|---|
| Aspirin | 20 mg/mL |
| Oxymetazoline HCl | 10 mg/mL |
| Dextromethorphan | 10 mg/mL |
| Diphenhydramine HCl | 5 mg/mL |
| Phenylephrine HCl | 10 mg/mL |
| Ibuprofen | 20 mg/mL |
| Hemoglobin | 20 mg/mL |
| Saline nasal spray | 10% |
| Mucin | 5% |

A concentration expressed herein as “% w/v” means weight per volume percent, which is equivalent to g solute per 0.1 L total volume, or g solute per 100 mL total volume.

The following definitions are to be used for the interpretation of the claims and specification. As used herein, the terms “comprises”, “comprising”, “includes”, “including”, “has”, “having”, “contains”, “containing”, or any other variation thereof, are intended to be non-exclusive. In other words, a composition, a mixture, process, method, or article that comprises a list of elements is not necessarily limited to only those elements but can include other elements not expressly listed or inherent in such composition, mixture, process, method, or article. Additionally, the terms “exemplary” and “example” are used herein to mean “serving as an example, instance or illustration.” Any embodiment described herein as “exemplary” is not necessarily to be construed as preferred or advantageous over other embodiments. The terms “at least one” and “one or more” are understood to include any integral number greater than or equal to one, i.e. one, two, three, four, etc. The term “a plurality” are understood to include any integral number greater than or equal to two, i.e. two, three, four, five, etc. “At least one of” as used herein in connection with a list means that the list is inclusive of each element individually, as well as combinations of two or more elements of the list, and combinations of at least one element of the list with like elements not named.

References in the specification to “one embodiment,” “an embodiment,” “some embodiments”, etc., indicate that the embodiment described can include a particular feature, structure, or characteristic, but every embodiment may or may not include the particular feature, structure, or characteristic. Moreover, such phrases do not necessarily refer to the same embodiment. When a particular feature, structure, or characteristic is described in connection with an embodiment, it is submitted that it is within the knowledge of one skilled in the art to include such feature, structure, or characteristic in connection with other embodiments whether or not such combination is explicitly described.

“At least one of” in connection with a list means that the list is inclusive of each element individually, as well as combinations of two or more elements of the list, as well as combinations of at least one element of the list with like elements not named.

The terms “about”, “substantially”, “approximately”, “circa”, and variations thereof are intended to include the degree of error associated with measurement of the particular quantity based upon the equipment available at the time of filing the application. For example, “about” a given value can include a range of ±10%, ±5%, or ±1% of the given value.

References to numerical ranges with lower and upper endpoints herein include all numbers subsumed within the range (including fractions), whether explicitly recited or not, as well as the endpoints of the range. Thus, “1 to 5” includes 1, 2, 3, 4, and 5 when referring to, for example, a number of elements, and can also include 1.5, 2, 2.75, 3.8, or any other decimal amount when referring to, for example, quantitative measurements.

Various embodiments of the method for collecting a liquid sample from the oral cavity and oropharyngeal area, and the rapid in vitro immunochromatographic assay and kit for the qualitative detection of analytes present in human oropharyngeal lavage, including SARS-CoV-2 virus antigen, are described herein with reference to related drawings. Alternative embodiments can be envisioned without departing from the scope of this disclosure.

The present disclosure includes the following numbered embodiments. The embodiments are numbered and refer to other embodiments by number, thus explicitly making logical connections between the embodiments. When a particular feature, structure, or characteristic is described in connection with an embodiment, it is within the ability of one skilled in the art to include such feature, structure, or characteristic in connection with other embodiments whether or not such combination is explicitly described elsewhere in the disclosure.

Embodiment 1. A method for collecting a liquid sample from the oral cavity and oropharyngeal area comprising: avoiding placing food, drink, gum, or tobacco products in the oral cavity for a least twenty minutes prior to collecting the liquid sample; pouring a non-alcoholic mouth rinse into the oral cavity; swishing and gargling the mouth rinse at least once to sweep inside of the oral cavity, including the inside of the cheeks, the tongue, and back of the throat, while not spitting or swallowing the mouth rinse; using a collection swab comprising a sponge head to scrub the inside of the oral cavity, including the inside of the cheeks, the tongue and back of the throat; inserting the collection swab under the tongue; holding the collection swab under the tongue for at least one minute without biting, sucking, or chewing on the sponge head to absorb the mouth rinse; and removing the collection swab from the oral cavity; wherein the sponge head is designed to absorb and discharge the entire volume of the mouth rinse.

Embodiment 1a. The method of embodiment 1, wherein the non-alcoholic mouth rinse is a composition comprising: a therapeutic antimicrobial mouth rinse; a salt; and a mucolytic agent.

Embodiment 1b. The method of embodiment 1a, comprising, based on the total volume of the composition: 12 to 62% w/v of the therapeutic antimicrobial mouth rinse; 5 to 25% w/v of the salt; and 2 to 8% w/v of the mucolytic agent.

Embodiment 1c. The method of embodiment 1a or 1b, wherein the therapeutic antimicrobial mouth rinse is at least one of cetylpyridinium chloride (CPC), chlorhexidine (CHX), or at least one essential oil selected from the group consisting of eucalyptol, menthol, methyl salicylate, and thymol.

Embodiment 1d. The method of any of embodiments 1a to 1c, wherein the salt comprises sodium chloride.

Embodiment 1e. The method of any of embodiments 1a to 1d, wherein the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

Embodiment 2. The method of embodiment 1, further comprising: inserting the collection swab, sponge-end first, into a sample collector wherein at least a section of the sample collector has an inner diameter or width smaller than the width of the sponge head; letting the collection swab inserted into the sample collector stand for at least one minute; pushing the sponge head of the collection swab up and down into the space defined by the inner wall of the section of the sample collector at least three times; pushing the sponge head against an inner wall of the sample collector to squeeze out a maximum amount of liquid sample; and sealing the sample collector with a cap.

Embodiment 3. The method of embodiment 2, wherein the cap comprises a nozzle for discharging drops of the liquid sample.

Embodiment 4. The method of embodiment 3, wherein the nozzle for discharging drops of the liquid sample is moveable so as to be open in a first position and closed in a second position.

Embodiment 5. A method for collecting a liquid sample from the oral cavity and oropharyngeal area of a human child comprising: avoiding placing food and drink in the oral cavity for a least 20 minutes prior to collecting the liquid sample; absorbing a non-alcoholic mouth rinse into the sponge head of a collection swab; using the collection swab to scrub the inside of the child's oral cavity, including the inside of the cheeks, the tongue and back of the throat; inserting the collection swab under the child's tongue; holding the collection swab under the child's tongue for at least one minute without biting, sucking, or chewing on the sponge head to absorb the mouth rinse; and removing the collection swab from the oral cavity; wherein the sponge head is designed to absorb and discharge the entire volume of the mouth rinse.

Embodiment 5a. The method of embodiment 5, wherein the non-alcoholic mouth rinse is a composition comprising: a therapeutic antimicrobial mouth rinse; a salt; and a mucolytic agent.

Embodiment 5b. The method of embodiment 5a, comprising, based on the total volume of the composition: 12 to 62% w/v of the therapeutic antimicrobial mouth rinse; 5 to 25% w/v of the salt; and 2 to 8% w/v of the mucolytic agent.

Embodiment 5c. The method of embodiment 5a or 5b, wherein the therapeutic antimicrobial mouth rinse is at least one of cetylpyridinium chloride (CPC), chlorhexidine (CHX), or at least one essential oil selected from the group consisting of eucalyptol, menthol, methyl salicylate, and thymol.

Embodiment 5d. The method of any of embodiments 5a to 5c, wherein the salt comprises sodium chloride.

Embodiment 5e. The method of any of embodiments 5a to 5d, wherein the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

Embodiment 6. A rapid in vitro immunochromatographic assay for the qualitative detection of analytes present in human oropharyngeal lavage comprising: collecting a liquid sample from the oral cavity and oropharyngeal area; adding a portion of the liquid sample to the sample port of a test card capable of immunochromatographic assay; and viewing the results on the test card; wherein a first colored band on the test card indicates a positive test result and a second colored control band indicates a valid test result.

Embodiment 7. The in vitro immunochromatographic assay of embodiment 6, wherein the test card is a lateral flow test card.

Embodiment 8. The in vitro immunochromatographic assay of embodiment 6, wherein the test card is a vertical flow test card.

Embodiment 9. The in vitro immunochromatographic assay of any of embodiments 6 to 8, wherein the collecting comprises: avoiding placing food, drink, gum, or tobacco products in the oral cavity for a least 20 minutes prior to collecting the liquid sample; pouring a non-alcoholic mouth rinse into the oral cavity; swishing and gargling the mouth rinse at least once to sweep inside of the oral cavity, including the inside of the cheeks, the tongue, and back of the throat, while not spitting or swallowing the mouth rinse; using a collection swab comprising a sponge head to scrub the inside of the oral cavity, including the inside of the cheeks, the tongue and back of the throat, and to absorb and discharge the mouth rinse; inserting the collection swab under the tongue; holding the collection swab under the tongue for at least one minute without biting, sucking, or chewing on the sponge head to absorb the mouth rinse; and removing the collection swab from the oral cavity; wherein the sponge head is designed to absorb and discharge the entire volume of the mouth rinse.

Embodiment 9a. The in vitro immunochromatographic assay of embodiment 9, wherein the non-alcoholic mouth rinse is a composition comprising: a therapeutic antimicrobial mouth rinse; a salt; and a mucolytic agent.

Embodiment 9b. The in vitro immunochromatographic assay of embodiment 9a, comprising, based on the total volume of the composition: 12 to 62% w/v of the therapeutic antimicrobial mouth rinse; 5 to 25% w/v of the salt; and 2 to 8% w/v of the mucolytic agent.

Embodiment 9c. The in vitro immunochromatographic assay of embodiment 9a or 9b, wherein the therapeutic antimicrobial mouth rinse is at least one of cetylpyridinium chloride (CPC), chlorhexidine (CHX), or at least one essential oil selected from the group consisting of eucalyptol, menthol, methyl salicylate, and thymol.

Embodiment 9d. The in vitro immunochromatographic assay of any of embodiments 9a to 9c, wherein the salt comprises sodium chloride.

Embodiment 9e. The in vitro immunochromatographic assay of any of embodiments 9a to 9d, wherein the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

Embodiment 10. The in vitro immunochromatographic assay of embodiment 9, wherein the collecting further comprises: inserting the collection swab, sponge-end first, into a sample collector wherein at least a section of the sample collector has an inner diameter or width smaller than the width of the sponge head; adding sample; letting the collection swab inserted into the sample collector stand for at least one minute; pushing the sponge head of the collection swab up and down into the space defined by the inner wall of the section of the sample collector at least three times; pushing the sponge head against an inner wall of the sample collector to squeeze out a maximum amount of liquid sample; and sealing the sample collector with a cap.

Embodiment 11. The method of embodiment 10, wherein the cap comprises a nozzle for discharging drops of the liquid sample.

Embodiment 12. The method of embodiment 11, wherein the nozzle for discharging drops of the liquid sample is moveable so as to be open in a first position and closed in a second position.

Embodiment 13. A rapid in vitro immunochromatographic assay for the qualitative detection of SARS CoV-2 virus antigen present in human oropharyngeal lavage, comprising: collecting a liquid sample from the oral cavity and oropharyngeal area; adding a portion of the liquid sample to the sample port of a test card capable of detecting the SARS CoV-2 virus antigen; and viewing test results on the test card; wherein a first colored band on the test card indicates a positive test result and a second colored control band indicates a valid test result.

Embodiment 14. The in vitro immunochromatographic assay of embodiment 13, wherein the test card is a lateral flow test card.

Embodiment 15. The in vitro immunochromatographic assay of embodiment 13, wherein the test card is a vertical flow test card.

Embodiment 16. The in vitro immunochromatographic assay of any of embodiments 13 to 15, wherein the collecting comprises: avoiding placing food, drink, gum, or tobacco products in the oral cavity for a least 20 minutes prior to collecting the liquid sample; pouring a non-alcoholic mouth rinse into the oral cavity; swishing and gargling the mouth rinse at least once to sweep inside of the oral cavity, including the inside of the cheeks, the tongue, and back of the throat, while not spitting or swallowing the mouth rinse; using a collection swab comprising a sponge head to scrub the inside of the oral cavity, including the inside of the cheeks, the tongue and back of the throat, and to absorb the mouth rinse; inserting the collection swab under the tongue; holding the collection swab under the tongue for at least one minute without biting, sucking, or chewing on the sponge head to absorb the mouth rinse; and removing the collection swab from the oral cavity; wherein the sponge head is designed to absorb and discharge the entire volume of the mouth rinse.

Embodiment 16a. The in vitro immunochromatographic assay of embodiment 16, wherein the non-alcoholic mouth rinse is a composition comprising: a therapeutic antimicrobial mouth rinse; a salt; and a mucolytic agent.

Embodiment 16b. The in vitro immunochromatographic assay of embodiment 16a, comprising, based on the total volume of the composition: 12 to 62% w/v of the therapeutic antimicrobial mouth rinse; 5 to 25% w/v of the salt; and 2 to 8% w/v of the mucolytic agent.

Embodiment 16c. The in vitro immunochromatographic assay of embodiment 16a or 16b, wherein the therapeutic antimicrobial mouth rinse is at least one of cetylpyridinium chloride (CPC), chlorhexidine (CHX), or at least one essential oil selected from the group consisting of eucalyptol, menthol, methyl salicylate, and thymol.

Embodiment 16d. The in vitro immunochromatographic assay of any of embodiments 16a to 16c, wherein the salt comprises sodium chloride.

Embodiment 16e. The in vitro immunochromatographic assay of any of embodiments 16a to 16d, wherein the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

Embodiment 17. The in vitro immunochromatographic assay of embodiment 16, wherein the collecting further comprises: inserting the collection swab, sponge-end first, into a sample collector wherein at least a section of the sample collector has an inner diameter or width smaller than the width of the sponge head; letting the collection swab inserted into the sample collector stand for at least one minute; pushing the sponge head of the collection swab up and down into the space defined by the inner wall of the section of the sample collector at least three times; pushing the sponge head against an inner wall of the sample collector to squeeze out a maximum amount of liquid sample; and sealing the sample collector with a cap.

Embodiment 18. The in vitro immunochromatographic assay of any of embodiments 13 to 17, wherein the test card comprises: a membrane; anti-SARS CoV-2 antibody-detection probe conjugates coated onto a capture zone of the membrane; anti-SARS CoV-2 antibodies coated onto a test zone of the membrane; a sample port; and a viewing port; wherein when the liquid sample contains SARS-CoV-2 viral antigens, the antigens form an antigen-antibody complex with the anti-SARS-CoV-2-detection probe conjugate, and the antigen-antibody complex is captured by anti-SARS CoV-2 antibodies coated onto the test zone to form a first colored band indicating a positive result; and wherein a second colored control band appears when the result is valid.

Embodiment 19. A kit for rapid in vitro immunochromatographic assay for the qualitative detection of SARS CoV-2 virus antigen present in human oropharyngeal lavage, the kit comprising: a collection swab comprising a sponge head; a non-alcoholic mouth rinse; a sample collector; a cap for the sample collector; a test card capable of detecting the SARS CoV-2 virus antigen; and instructions for conducting the assay.

Embodiment 19a. The kit of embodiment 19, wherein the non-alcoholic mouth rinse is a composition comprising: a therapeutic antimicrobial mouth rinse; a salt; and a mucolytic agent.

Embodiment 19b. The kit of embodiment 19a, wherein the non-alcoholic mouth rinse is obtained by dilution of a stock solution comprising, based on the total volume of the stock solution, 12 to 62% w/v of the therapeutic antimicrobial mouth rinse, 5 to 25% w/v of the salt, and 2 to 8% w/v of the mucolytic agent, with distilled water in a volume ratio of stock solution to distilled water of from about 1:2 to about 1:10.

Embodiment 19c. The kit of embodiment 19a or 19b, wherein the therapeutic antimicrobial mouth rinse is at least one of cetylpyridinium chloride (CPC), chlorhexidine (CHX), or at least one essential oil selected from the group consisting of eucalyptol, menthol, methyl salicylate, and thymol.

Embodiment 19d. The kit of any of embodiments 19a to 19c, wherein the salt comprises sodium chloride.

Embodiment 19e. The kit of any of embodiments 19a to 19d, wherein the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

Embodiment 20. The kit of embodiment 19, wherein the test card comprises: a membrane; anti-SARS CoV-2 antibody-detection probe conjugates coated onto a capture zone of the membrane; anti-SARS CoV-2 antibodies coated onto a test zone of the membrane; a sample port; and a viewing port; wherein when the liquid sample contains SARS-CoV-2 viral antigens, the antigens form an antigen-antibody complex with the anti-SARS-CoV-2-detection probe conjugate, and the antigen-antibody complex is captured by anti-SARS CoV-2 antibodies coated onto the test zone to form a first colored band indicating a positive result; and wherein a second colored control band appears when the result is valid.

Embodiment 21. A composition comprising: a therapeutic antimicrobial mouth rinse; a salt; and a mucolytic agent.

Embodiment 22. The composition of embodiment 21, wherein the composition is a stock solution comprising, based on the total volume of the stock solution: 12 to 62% w/v of the therapeutic antimicrobial mouth rinse; 5 to 25% w/v of the salt; and 2 to 8% w/v of the mucolytic agent.

Embodiment 23. The composition of embodiment 21 or 22, wherein the therapeutic antimicrobial mouth rinse comprises an antimicrobial that is at least one of cetylpyridinium chloride (CPC), chlorhexidine (CHX), or at least one essential oil selected from the group consisting of eucalyptol, menthol, methyl salicylate, and thymol.

Embodiment 24. The composition of any of embodiments 21 to 23, wherein the salt comprises sodium chloride.

Embodiment 25. The composition of any of embodiments 21 to 24, wherein the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

Embodiment 26. The composition of any of embodiments 21 to 25, wherein: the therapeutic antimicrobial mouth rinse comprises an antimicrobial that is at least one of cetylpyridinium chloride (CPC), chlorhexidine (CHX), or at least one essential oil selected from the group consisting of eucalyptol, menthol, methyl salicylate, and thymol; the salt comprises sodium chloride; and the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

Embodiment 27. The method of embodiment 1, wherein the non-alcoholic mouth rinse is a composition comprising: a therapeutic antimicrobial mouth rinse; a salt; and a mucolytic agent.

Embodiment 28. The method of embodiment 5, wherein the non-alcoholic mouth rinse is a composition comprising: a therapeutic antimicrobial mouth rinse; a salt; and a mucolytic agent.

Embodiment 28a. The method of embodiment 28, wherein the non-alcoholic mouth rinse is obtained by dilution of a stock solution comprising, based on the total volume of the stock solution, 12 to 62% w/v of the therapeutic antimicrobial mouth rinse, 5 to 25% w/v of the salt, and 2 to 8% w/v of the mucolytic agent, with distilled water in a volume ratio of stock solution to distilled water of from about 1:2 to about 1:10.

Embodiment 29. The in vitro immunochromatographic assay of embodiment 9, wherein the non-alcoholic mouth rinse is a composition comprising: a therapeutic antimicrobial mouth rinse; a salt; and a mucolytic agent.

Embodiment 29a. The in vitro immunochromatographic assay of embodiment 29, wherein the non-alcoholic mouth rinse is obtained by dilution of a stock solution comprising, based on the total volume of the stock solution, 12 to 62% w/v of the therapeutic antimicrobial mouth rinse, 5 to 25% w/v of the salt, and 2 to 8% w/v of the mucolytic agent, with distilled water in a volume ratio of stock solution to distilled water of from about 1:2 to about 1:10.

Embodiment 30. The rapid in vitro immunochromatographic assay of embodiment 13, wherein the non-alcoholic mouth rinse is a composition comprising: a therapeutic antimicrobial mouth rinse; a salt; and a mucolytic agent.

Embodiment 30a. The rapid in vitro immunochromatographic assay of embodiment 30, wherein the non-alcoholic mouth rinse is obtained by dilution of a stock solution comprising, based on the total volume of the stock solution, 12 to 62% w/v of the therapeutic antimicrobial mouth rinse, 5 to 25% w/v of the salt, and 2 to 8% w/v of the mucolytic agent, with distilled water in a volume ratio of stock solution to distilled water of from about 1:2 to about 1:10.

Embodiment 31. The kit of embodiment 19, wherein the non-alcoholic mouth rinse is a composition comprising: a therapeutic antimicrobial mouth rinse; a salt; and a mucolytic agent.

Embodiment 31a. The kit of embodiment 21, wherein the non-alcoholic mouth rinse is obtained by dilution of a stock solution comprising, based on the total volume of the stock solution, 12 to 62% w/v of the therapeutic antimicrobial mouth rinse, 5 to 25% w/v of the salt, and 2 to 8% w/v of the mucolytic agent, with distilled water in a volume ratio of stock solution to distilled water of from about 1:2 to about 1:10.

Embodiment 32. A non-alcoholic mouth rinse obtained by diluting the stock solution of embodiment 22 with distilled water in a volume ratio of stock solution to distilled water of from about 1:2 to about 1:10.

While preferred embodiments of the method for collecting a liquid sample from the oral cavity and oropharyngeal area, and the rapid in vitro immunochromatographic assay and kit for the qualitative detection of analytes present in human oropharyngeal lavage, including SARS-CoV-2 virus antigen, are disclosed herein, those skilled in the art, both now and in the future, may make various improvements and enhancements which still fall within the scope of the claims which follow. Thus, these claims should be construed to encompass unnamed improvements and enhancements in the method of claimed methods, assays, and kits.

What is claimed is:

1. A method for collecting a liquid sample from the oral cavity and oropharyngeal area comprising:
avoiding placing food, drink, gum, or tobacco products in the oral cavity for a least twenty minutes prior to collecting the liquid sample;
pouring a non-alcoholic mouth rinse into the oral cavity;
swishing and gargling the mouth rinse at least once to sweep inside of the oral cavity, including the inside of the cheeks, the tongue, and back of the throat, while not spitting or swallowing the mouth rinse;
using a collection swab comprising a sponge head to scrub the inside of the oral cavity, including the inside of the cheeks, the tongue and back of the throat;
inserting the collection swab under the tongue;
holding the collection swab under the tongue for at least one minute without biting, sucking, or chewing on the sponge head to absorb the mouth rinse; and
removing the collection swab from the oral cavity;
wherein the sponge head is designed to absorb and discharge the entire volume of the mouth rinse.

2. The method of claim 1, further comprising:
inserting the collection swab, sponge-end first, into a sample collector wherein at least a section of the sample collector has an inner diameter or width smaller than the width of the sponge head;
letting the collection swab inserted into the sample collector stand for at least one minute;
pushing the sponge head of the collection swab up and down into the space defined by the inner wall of the section of the sample collector at least three times;
pushing the sponge head against an inner wall of the sample collector to squeeze out a maximum amount of liquid sample; and
sealing the sample collector with a cap.

3. The method of claim 2, wherein the cap comprises a nozzle for discharging drops of the liquid sample.

4. The method of claim 3, wherein the nozzle for discharging drops of the liquid sample is moveable so as to be open in a first position and closed in a second position.

5. A method for collecting a liquid sample from the oral cavity and oropharyngeal area of a human child comprising:
avoiding placing food and drink in the oral cavity for a least 20 minutes prior to collecting the liquid sample;
absorbing a non-alcoholic mouth rinse into the sponge head of a collection swab;
using the collection swab to scrub the inside of the child's oral cavity, including the inside of the cheeks, the tongue and back of the throat;
inserting the collection swab under the child's tongue;
holding the collection swab under the child's tongue for at least one minute without biting, sucking, or chewing on the sponge head to absorb the mouth rinse; and
removing the collection swab from the oral cavity;
wherein the sponge head is designed to absorb and discharge the entire volume of the mouth rinse.

6. A rapid in vitro immunochromatographic assay for the qualitative detection of analytes present in human oropharyngeal lavage comprising:
collecting a liquid sample from the oral cavity and oropharyngeal area;
adding a portion of the liquid sample to the sample port of a test card capable of immunochromatographic assay; and
viewing the results on the test card;
wherein a first colored band on the test card indicates a positive test result and a second colored control band indicates a valid test result.

7. The in vitro immunochromatographic assay of claim 6, wherein the test card is a lateral flow test card.

8. The in vitro immunochromatographic assay of claim 6, wherein the test card is a vertical flow test card.

9. The in vitro immunochromatographic assay of claim 6, wherein the collecting comprises:
avoiding placing food, drink, gum, or tobacco products in the oral cavity for a least 20 minutes prior to collecting the liquid sample;
pouring a non-alcoholic mouth rinse into the oral cavity;
swishing and gargling the mouth rinse at least once to sweep inside of the oral cavity, including the inside of the cheeks, the tongue, and back of the throat, while not spitting or swallowing the mouth rinse;
using a collection swab comprising a sponge head to scrub the inside of the oral cavity, including the inside of the cheeks, the tongue and back of the throat;
inserting the collection swab under the tongue;
holding the collection swab under the tongue for at least one minute without biting, sucking, or chewing on the sponge head to absorb the mouth rinse; and
removing the collection swab from the oral cavity;
wherein the sponge head is designed to absorb and discharge the entire volume of the mouth rinse.

10. The in vitro immunochromatographic assay of claim 9, wherein the collecting further comprises:
inserting the collection swab, sponge-end first, into a sample collector wherein at least a section of the sample collector has an inner diameter or width smaller than the width of the sponge head;
letting the collection swab inserted into the sample collector stand for at least one minute;
pushing the sponge head of the collection swab up and down into the space defined by the inner wall of the section of the sample collector at least three times;
pushing the sponge head against an inner wall of the sample collector to squeeze out a maximum amount of liquid sample; and
sealing the sample collector with a cap.

11. The method of claim 10, wherein the cap comprises a nozzle for discharging drops of the liquid sample.

12. The method of claim 11, wherein the nozzle for discharging drops of the liquid sample is moveable so as to be open in a first position and closed in a second position.

13. A rapid in vitro immunochromatographic assay for the qualitative detection of SARS-CoV-2 virus antigen present in human oropharyngeal lavage, comprising:
collecting a liquid sample from the oral cavity and oropharyngeal area;
adding a portion of the liquid sample to the sample port of a test card capable of detecting the SARS-CoV-2 virus antigen; and
viewing test results on the test card;
wherein a first colored band on the test card indicates a positive test result and a second colored control band indicates a valid test result.

14. The in vitro immunochromatographic assay of claim 13, wherein the test card is a lateral flow test card.

15. The in vitro immunochromatographic assay of claim 13, wherein the test card is a vertical flow test card.

16. The in vitro immunochromatographic assay of claim 13, wherein the collecting comprises:
avoiding placing food, drink, gum, or tobacco products in the oral cavity for a least 20 minutes prior to collecting the liquid sample;
pouring a non-alcoholic mouth rinse into the oral cavity;
swishing and gargling the mouth rinse at least once to sweep inside of the oral cavity, including the inside of the cheeks, the tongue, and back of the throat, while not spitting or swallowing the mouth rinse;
using a collection swab comprising a sponge head to scrub the inside of the oral cavity, including the inside of the cheeks, the tongue and back of the throat;
inserting the collection swab under the tongue;
holding the collection swab under the tongue for at least one minute without biting, sucking, or chewing on the sponge head to absorb the mouth rinse; and
removing the collection swab from the oral cavity;
wherein the sponge head is designed to absorb and discharge the entire volume of the mouth rinse.

17. The in vitro immunochromatographic assay of claim 16, wherein the collecting further comprises:
inserting the collection swab, sponge-end first, into a sample collector wherein at least a section of the sample collector has an inner diameter or width smaller than the width of the sponge head;
letting the collection swab inserted into the sample collector stand for at least one minute;
pushing the sponge head of the collection swab up and down into the space defined by the inner wall of the section of the sample collector at least three times;
pushing the sponge head against an inner wall of the sample collector to squeeze out a maximum amount of liquid sample; and
sealing the sample collector with a cap.

18. The in vitro immunochromatographic assay of claim 13, wherein the test card comprises:
a membrane;
anti-SARS CoV-2 antibody-detection probe conjugates coated onto a capture zone of the membrane;
anti-SARS CoV-2 antibodies coated onto a test zone of the membrane;
a sample port; and
a viewing port;
wherein when the liquid sample contains SARS-CoV-2 viral antigens, the antigens form an antigen-antibody complex with the anti-SARS-CoV-2-detection probe conjugate, and the antigen-antibody complex is captured by anti-SARS CoV-2 antibodies coated onto the test zone to form a first colored band indicating a positive result; and
wherein a second colored control band appears when the result is valid.

19. A kit for rapid in vitro immunochromatographic assay for the qualitative detection of SARS-CoV-2 virus antigen present in human oropharyngeal lavage, the kit comprising:
a collection swab comprising a sponge head;
a non-alcoholic mouth rinse;
a sample collector;
a cap for the sample collector;
a test card capable of detecting the SARS-CoV-2 virus antigen; and
instructions for conducting the assay.

20. The kit of claim 19, wherein the test card comprises:
a membrane;
anti-SARS CoV-2 antibody-detection probe conjugates coated onto a capture zone of the membrane;
anti-SARS CoV-2 antibodies coated onto a test zone of the membrane;
a sample port; and
a viewing port;
wherein when the liquid sample contains SARS-CoV-2 viral antigens, the antigens form an antigen-antibody complex with the anti-SARS-CoV-2-detection probe conjugate, and the antigen-antibody complex is captured by anti-SARS CoV-2 antibodies coated onto the test zone to form a first colored band indicating a positive result; and
wherein a second colored control band appears when the result is valid.

21. A composition comprising:
a therapeutic antimicrobial mouth rinse;
a salt; and
a mucolytic agent.

22. The composition of claim 21, wherein the composition is a stock solution comprising, based on the total volume of the stock solution:
12 to 62% w/v of the therapeutic antimicrobial mouth rinse;
5 to 25% w/v of the salt; and
2 to 8% w/v of the mucolytic agent.

23. The composition of claim 21, wherein the therapeutic antimicrobial mouth rinse comprises an antimicrobial that is at least one of cetylpyridinium chloride (CPC), chlorhexidine (CHX), or at least one essential oil selected from the group consisting of eucalyptol, menthol, methyl salicylate, and thymol.

24. The composition of claim 21, wherein the salt comprises sodium chloride.

25. The composition of claim 21, wherein the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

26. The composition of claim 21, wherein:
the therapeutic antimicrobial mouth rinse is at least one of cetylpyridinium chloride (CPC), chlorhexidine (CHX), or at least one essential oil selected from the group consisting of eucalyptol, menthol, methyl salicylate, and thymol;
the salt comprises sodium chloride; and
the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

27. The method of claim 1, wherein the non-alcoholic mouth rinse is a composition comprising:
a therapeutic antimicrobial mouth rinse;
a salt; and
a mucolytic agent.

28. The method of claim 5, wherein the non-alcoholic mouth rinse is a composition comprising:
a therapeutic antimicrobial mouth rinse;
a salt; and
a mucolytic agent.

29. The in vitro immunochromatographic assay of claim 9, wherein the non-alcoholic mouth rinse is a composition comprising:
a therapeutic antimicrobial mouth rinse;
a salt; and
a mucolytic agent.

30. The rapid in vitro immunochromatographic assay of claim 13, wherein the non-alcoholic mouth rinse is a composition comprising:
a therapeutic antimicrobial mouth rinse;
a salt; and
a mucolytic agent.

31. The kit of claim 19, wherein the non-alcoholic mouth rinse is a composition comprising:
a therapeutic antimicrobial mouth rinse;
a salt; and
a mucolytic agent.

32. A non-alcoholic mouth rinse obtained by diluting the stock solution of claim 22 with distilled water in a volume ratio of stock solution to distilled water of from about 1:2 to about 1:10.
